(19) United States
(12) Patent Application Publication
(10) Pub. No.: US 2026/0185107 A1
(43) Pub. Date: Jul. 2, 2026

(54) COMPOSITIONS AND METHODS FOR IMPROVED BIOPRODUCTION

(71) Applicants: Nitto Denko Corporation, Osaka (JP); Safion Renewables Inc., Cos Cob, CT (US)

(72) Inventors: Andrew John BORCHERT, Arvada, CO (US); Lauren Ann Riley DRUMM, Lafayette, CO (US)

(21) Appl. No.: 19/327,989

(22) Filed: Sep. 12, 2025

Related U.S. Application Data
(60) Provisional application No. 63/714,741, filed on Oct. 31, 2024.

(51) Int. Cl. C12N 15/52; C12N 1/20

(52) U.S. Cl. CPC C12N 15/52; C12N 1/20; C12Y 203/01184; C12Y 204/01012

(57) ABSTRACT

Provided herein are compositions and methods for engineering a bacterium comprising a reduced capacity for cellulose formation, a reduced capacity for foaming behavior, or an increased capacity for cell density for enhancing bioprocessing outcomes associated with the fermentative production of industrially relevant bioproducts.

### CROSS REFERENCE TO RELATED APPLICATIONS

This application claims the benefit of U.S. Provisional Application No. 63/714,741, filed Oct. 31, 2024.

### REFERENCE TO SEQUENCE LISTING

The present application is being filed along with a Sequence Listing in electronic format. The Sequence Listing is provided as a file entitled NDTUS011.xml, created on Feb. 12, 2026 which is approximately 21,051 bytes in size. The information in the electronic format of the Sequence Listing is incorporated herein by reference in its entirety.

### BACKGROUND

Fermentation of bacteria can be used to yield bioproducts produced by the bacteria. Acetic acid bacteria are widespread and versatile organisms that can be used to produce bioproducts.

### SUMMARY

Engineered bacterium which overcome limitations associated with scaled bioproduction may improve bacterial production of industrially relevant bioproducts. Provided herein, in some embodiments, are compositions and methods for enhancing bioprocessing and culture handling outcomes associated with fermentation-based production of industrially relevant bioproducts. The compositions and methods provided herein comprise an engineered bacterium that may reduce cellulose production, reduce foam formation, and/or improve cell density (e.g., improve volumetric productivity) during fermentation.

Provided herein, in some embodiments, is an engineered bacterium that comprises (a) a reduced capacity for cellulose formation compared to a capacity for cellulose formation of a wild type acetic acid bacterium (AAB), (b) a reduced capacity for foaming behavior compared to a foaming behavior of the wild type AAB, (c) an increased capacity for cell density compared to a capacity for cell density of the wild type AAB, or (d) a combination thereof.

In some embodiments, the engineered bacterium comprises a reduced capacity for cellulose formation compared to a capacity for cellulose formation of the wild type AAB. In some embodiments, the engineered bacterium comprises a modification of at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase. In some embodiments, the modification comprises knockout, disruption, truncation, knockdown, or inhibition of the at least one gene.

In some embodiments, the engineered bacterium comprises a reduced capacity for cellulose formation compared to a capacity for cellulose formation of the wild type AAB. In some embodiments, the engineered bacterium comprises a modification of at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase QsdR1. In some embodiments, the modification comprises enhanced expression of the at least one gene, heterologous expression of the at least one gene, or enhanced activity of a gene product associated with the at least one gene.

In some embodiments, the engineered bacterium comprises a reduced capacity for foaming behavior compared to a foaming behavior of the wild type AAB. In some embodiments, the engineered bacterium comprises a reduced capacity for expression of GinA compared to a capacity for expression of GinA of the wild type acetic acid bacterium. In some embodiments, the engineered bacterium comprises a modification of at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator. In some embodiments, the modification comprises knockout, disruption, truncation, knockdown, or inhibition of the at least one gene.

In some embodiments, the engineered bacterium comprises a reduced capacity for foaming behavior compared to a foaming behavior of the wild type AAB. In some embodiments, the engineered bacterium comprises a modification of a gene encoding N-Acyl-homoserine lactone acylase GqqA or a gene encoding N-Acyl-homoserine lactone lactonase QsdR1. In some embodiments, the modification comprises enhanced expression of the at least one gene, heterologous expression of the at least one gene, or enhanced activity of a gene product associated with the at least one gene.

In some embodiments, the engineered bacterium comprises an increased capacity for cell density compared to a capacity for cell density of a wild type AAB. In some embodiments, the engineered bacterium comprises a modification of at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein. In some embodiments, the modification comprises knockout, disruption, truncation, knockdown, or inhibition of the at least one gene.

In some embodiments, the engineered bacterium comprises an increased capacity for cell density compared to a capacity for cell density of a wild type AAB. In some embodiments, the engineered bacterium comprises a modification of a gene encoding N-Acyl-homoserine lactone acylase GqqA or a gene encoding N-Acyl-homoserine lactone lactonase QsdR1. In some embodiments, the modification comprises enhanced expression of the at least one gene, heterologous expression of the at least one gene, or enhanced activity of a gene product associated with the at least one gene.

Provided herein, in some embodiments, is an engineered bacterium. In some embodiments, the engineered bacterium comprises a modification of (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase. In some embodiments, the engineered bacterium comprises a modification of (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase QsdR1. In some embodiments, the engineered bacterium comprises a modification of (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator. In some embodiments, the engineered bacterium comprises a modification of (d) at least one gene selected from the group consisting of: a gene encoding N-Acyl-homoserine lactone acylase GqqA and a gene encoding N-Acyl-homoserine lactone lactonase QsdR1. In some embodiments, the engineered bacterium comprises a modification of (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein. In some embodiments, the engineered bacterium comprises a modification of (f) a combination of (a), (b), (c), (d), or (e) thereof.

Provided herein, in some embodiments, is an engineered bacterium that comprises a modification of: (a) at least one gene selected from the group consisting of: a gene encoding a bacterial cellulose synthesis protein, a gene encoding phosphoglucomutase, a gene encoding a UTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase QsdR1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; (d) at least one gene selected from the group consisting of: a gene encoding N-Acyl-homoserine lactone acylase GqqA and a gene encoding N-Acyl-homoserine lactone lactonase QsdR1; (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein; or (f) a combination thereof. In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; and (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase QsdR1.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; and (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; and (d) at least one gene selected from the group consisting of: a gene encoding N-Acyl-homoserine lactone acylase GqqA and a gene encoding N-Acyl-homoserine lactone lactonase QsdR1.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator.

In some embodiments, the engineered bacterium comprises a modification of: (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; and (d) at least one gene selected from the group consisting of: a gene encoding N-Acyl-homoserine lactone acylase GqqA and a gene encoding N-Acyl-homoserine lactone lactonase Qsdr1.

In some embodiments, the engineered bacterium comprises a modification of: (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; and (d) at least one gene selected from the group consisting of: a gene encoding N-Acyl-homoserine lactone acylase GqqA and a gene encoding N-Acyl-homoserine lactone lactonase Qsdr1.

In some embodiments, the engineered bacterium comprises a modification of: (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (d) at least one gene selected from the group consisting of: a gene encoding N-Acyl-homoserine lactone acylase GqqA and a gene encoding N-Acyl-homoserine lactone lactonase Qsdr1; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; and (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; and (d) at least one gene selected from the group consisting of: a gene encoding N-Acyl-homoserine lactone acylase GqqA and a gene encoding N-Acyl-homoserine lactone lactonase Qsdr1.

n some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; and (d) at least one gene selected from the group consisting of: a gene encoding N-Acyl-homoserine lactone acylase GqqA and a gene encoding N-Acyl-homoserine lactone lactonase Qsdr1.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (d) at least one gene selected from the group consisting of: a gene encoding N-Acyl-homoserine lactone acylase GqqA and a gene encoding N-Acyl-homoserine lactone lactonase Qsdr1; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; and (d) at least one gene selected from the group consisting of: a gene encoding N-Acyl-homoserine lactone acylase GqqA and a gene encoding N-Acyl-homoserine lactone lactonase Qsdr1.

In some embodiments, the engineered bacterium comprises a modification of: (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (d) at least one gene selected from the group consisting of: a gene encoding N-Acyl-homoserine lactone acylase GqqA and a gene encoding N-Acyl-homoserine lactone lactonase Qsdr1; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; (d) at least one gene selected from the group consisting of: a gene encoding N-Acyl-homoserine lactone acylase GqqA and a gene encoding N-Acyl-homoserine lactone lactonase Qsdr1; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; and (d) at least one gene selected from the group consisting of: a gene encoding N-Acyl-homoserine lactone acylase GqqA and a gene encoding N-Acyl-homoserine lactone lactonase Qsdr1.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (d) at least one gene selected from the group consisting of: a gene encoding N-Acyl-homoserine lactone acylase GqqA and a gene encoding N-Acyl-homoserine lactone lactonase Qsdr1; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; (d) at least one gene selected from the group consisting of: a gene encoding N-Acyl-homoserine lactone acylase GqqA and a gene encoding N-Acyl-homoserine lactone lactonase Qsdr1; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; (d) at least one gene selected from the group consisting of: a gene encoding N-Acyl-homoserine lactone acylase GqqA and a gene encoding N-Acyl-homoserine lactone lactonase Qsdr1; and (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises (a) knockout, disruption, truncation, knockdown, or inhibition of at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase. In some embodiments, the engineered bacterium comprises (b) heterologous expression of, increased expression of, or increased activity of a gene product associated with at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1. In some embodiments, the engineered bacterium comprises (c) knockout, disruption, truncation, knockdown, or inhibition of at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator. In some embodiments, the engineered bacterium comprises (d) heterologous expression of, increased expression of, or increased activity of the gene product associated with at least one gene selected from the group consisting of: a gene encoding N-Acyl-homoserine lactone acylase GqqA and a gene encoding N-Acyl-homoserine lactone lactonase Qsdr1. In some embodiments, the engineered bacterium comprises (e) knockout, disruption, truncation, knockdown, or inhibition of at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises (a) knockout, disruption, truncation, knockdown, or inhibition of at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) heterologous expression of, increased expression of, or increased activity of a gene product associated with at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) knockout, disruption, truncation, knockdown, or inhibition of at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; (d) heterologous expression of, increased expression of, or increased activity of the gene product associated with at least one gene selected from the group consisting of: a gene encoding N-Acyl-homoserine lactone acylase GqqA and a gene encoding N-Acyl-homoserine lactone lactonase Qsdr1; or (e) knockout, disruption, truncation, knockdown, or inhibition of at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some embodiments, the engineered bacterium comprises the reduced capacity for cellulose formation by at least about 50% compared to the capacity for cellulose formation of the wild type AAB. In some embodiments, the engineered bacterium comprises the reduced capacity for cellulose formation by at least about 60% compared to the capacity for cellulose formation of the wild type AAB. In some embodiments, the engineered bacterium comprises the reduced capacity for cellulose formation by at least about 70% compared to the capacity for cellulose formation of the wild type AAB. In some embodiments, the engineered bacterium comprises the reduced capacity for cellulose formation by at least about 80% compared to the capacity for cellulose formation of the wild type AAB. In some embodiments, the engineered bacterium comprises the reduced capacity for cellulose formation by at least about 90% compared to the capacity for cellulose formation of the wild type AAB. In some embodiments, the engineered bacterium comprises the reduced capacity for cellulose formation by at least about 95% compared to the capacity for cellulose formation of the wild type AAB. In some embodiments, the engineered bacterium comprises the reduced capacity for cellulose formation by at least about 100% compared to the capacity for cellulose formation of the wild type AAB.

In some embodiments, the engineered bacterium comprises the reduced capacity for foaming behavior by at least about 5% compared to a foaming behavior of the wild type AAB. In some embodiments, the engineered bacterium comprises the reduced capacity for foaming behavior by at least about 6% compared to a foaming behavior of the wild type AAB. In some embodiments, the engineered bacterium comprises the reduced capacity for foaming behavior by at least about 7% compared to a foaming behavior of the wild type AAB. In some embodiments, the engineered bacterium comprises the reduced capacity for foaming behavior by at least about 8% compared to a foaming behavior of the wild type AAB. In some embodiments, the engineered bacterium comprises the reduced capacity for foaming behavior by at least about 9% compared to a foaming behavior of the wild type AAB. In some embodiments, the engineered bacterium comprises the reduced capacity for foaming behavior by at least about 10% compared to a foaming behavior of the wild type AAB. [0041] In some embodiments, the engineered bacterium comprises the increased capacity for cell density by at least about 5% compared to a capacity for cell density of the wild type AAB. In some embodiments, the engineered bacterium comprises the increased capacity for cell density by at least about 6% compared to a capacity for cell density of the wild type AAB. In some embodiments, the engineered bacterium comprises the increased capacity for cell density by at least about 7% compared to a capacity for cell density of the wild type AAB. In some embodiments, the engineered bacterium comprises the increased capacity for cell density by at least about 8% compared to a capacity for cell density of the wild type AAB. In some embodiments, the engineered bacterium comprises the increased capacity for cell density by at least about 9% compared to a capacity for cell density of the wild type AAB. In some embodiments, the engineered bacterium comprises the increased capacity for cell density by at least about 10% compared to a capacity for cell density of the wild type AAB. [0042] In some embodiments, the capacities for cellulose formation are measurable by detection of cellulose produced by the bacterium in a bioreactor. In some embodiments, the capacities for foaming behavior are measurable by detection of a foam layer produced by the bacterium in a bioreactor. In some embodiments, the capacities for cell density are measured by detection of cell density of the bacterium in a bioreactor. In some embodiments, the reduced capacity for cellulose formation, reduced capacity for foaming behavior, or increased capacity for cell density are measured under culture conditions that comprise a temperature of 30° C.; a growth time of at least 72 hours; a pH between 3 and 7; a culture media that comprise a carbon source, a nitrogen source, minerals, amino acids, or vitamins, or a combination thereof; or a combination thereof.

In some embodiments, the engineered bacterium is an acetic acid bacterium. In some embodiments, the engineered bacterium is of a genus Acetobacter, a genus Gluconacetobacter, a genus Gluconobacter, or a genus Komagataeibacter. In some embodiments, the engineered bacterium is Komagataeibacter europaeus LMG 1521.

In some embodiments, the at least one gene is a natively expressed gene. In some embodiments, the modification comprises knockout, disruption, truncation, knockdown, or inhibition of the at least one gene. In some embodiments, the modification results in increased expression of the at least one gene or increased activity of the gene product associated with the at least one gene. In some embodiments, the at least one gene is a non-natively expressed gene. In some embodiments, the modification comprises heterologous expression of the at least one gene.

Provided herein, in some embodiments, is a method of making a bioproduct that comprises growing the engineered bacterium described herein. In some embodiments, the bioproduct is acetic acid, L-sorbose, gluconic acid, 2-keto-D-gluconate, 5-keto-D-gluconate, dihydroxyacetone (DHA), cellulose, or acetan. In some embodiments, the bioproduct is acetic acid. In some embodiments, the method comprises growing the engineered bacterium under culture conditions that comprise a temperature of 30° C.; a growth time of at least 72 hours; a pH between 3 and 7; a culture media that comprises a carbon source, a nitrogen source, minerals, amino acids, or vitamins, or a combination thereof; or a combination thereof.

Provided herein, in some embodiments, is a method of making the engineered bacterium described herein that comprises modifying in a bacterium: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; (d) at least one gene selected from the group consisting of: a gene encoding N-Acyl-homoserine lactone acylase GqqA and a gene encoding N-Acyl-homoserine lactone lactonase Qsdr1; (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein; or (f) a combination thereof.

Provided herein, in some embodiments, is a method of making an engineered bacterium. In some embodiments, the method comprises modifying in a bacterium (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase. In some embodiments, the method comprises modifying in a bacterium (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1. In some embodiments, the method comprises modifying in a bacterium (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator. In some embodiments, the method comprises modifying in a bacterium (d) at least one gene selected from the group consisting of: a gene encoding N-Acyl-homoserine lactone acylase GqqA and a gene encoding N-Acyl-homoserine lactone lactonase Qsdr1. In some embodiments, the method comprises modifying in a bacterium (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein. In some embodiments, the method comprises modifying in a bacterium (f) a combination of (a), (b), (c), (d), or (e) thereof.

Provided herein, in some embodiments, is a method of making an engineered bacterium that comprises modifying in a bacterium: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; (d) at least one gene selected from the group consisting of: a gene encoding N-Acyl-homoserine lactone acylase GqqA and a gene encoding N-Acyl-homoserine lactone lactonase Qsdr1; (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein; or (f) a combination thereof. [0049] In some embodiments, the at least one gene is a natively expressed gene. In some embodiments, the modifying comprises knocking out, disrupting, knocking down, or inhibiting the at least one gene. In some embodiments, the modifying comprises increasing expression of the at least one gene or increasing activity of a gene product associated with the at least one gene.

In some embodiments, the at least one gene is a non-natively expressed gene. In some embodiments, the modifying comprises heterologous expression of the at least one gene.

In some embodiments, the modifying comprises modification with a CRISPR/Cas system, a homologous recombination system, a phage recombinase system, a phage integrase system, or a transposase system.

In some embodiments, the bacterium is an acetic acid bacterium. In some embodiments, the bacterium is of a genus Acetobacter, a genus Gluconacetobacter, a genus Gluconobacter, or a genus Komagataeibacter. In some embodiments, the bacterium is Komagataeibacter europaeus LMG 1521.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows a gene-modification vector that can be used to make an engineered bacterium. The vector comprises an aminoglycoside phosphotransferase gene (aphA1) conferring kanamycin resistance, a levansucrase gene (sacB) conferring sucrose sensitivity, a class A tetracycline resistance protein (tetA) gene conferring tetracycline resistance and fusaric acid sensitivity, a β-galactosidase gene (lacZ) conferring the ability to catabolize 5-bromo-4-chloro-3-indolyl-β-D-galactopyranoside (X-Gal, which facilitates blue/white colony screening), a lactose permease gene (lacY, which facilitates transportation of X-Gal into the engineered bacterium), and upstream and downstream homologous nucleotide sequences (700-800 bp each) corresponding to the gene or sequence of interest targeted for mutation and a genetic modification sequence flanked by the upstream and downstream homologous nucleotide sequences. The genetic modification sequence causes modification of a gene or sequence of interest (e.g., truncation of wcaE) in an engineered bacterium.

FIG. 2A shows an expanded view of a gene-modification vector that can be used to make an engineered bacterium that comprises a deletion of bcsABCD. The vector comprises at least upstream and downstream homologous nucleotide sequences (700-800 bp each) corresponding to the gene or sequence of interest targeted for mutation and a genetic modification sequence (e.g., “ΔbcsABCD”) flanked by the upstream and downstream homologous nucleotide sequences. In this vector, the genetic modification sequence is empty. The upstream homologous nucleotide sequence (e.g., “Upstream Homology”) comprises homology to an endoglucanase coding sequence and is upstream of the gene of interest targeted for deletion (e.g., bcsABCD). The downstream homologous nucleotide sequence (e.g., “Downstream Homology”) comprises homology to a bgIX coding sequence and is downstream of the gene of interest targeted for deletion (e.g., bcsABCD). The vector may also comprise an aminoglycoside phosphotransferase gene (aphA1) conferring kanamycin resistance, a levansucrase gene (sacB) conferring sucrose sensitivity, a class A tetracycline resistance protein (tetA) gene conferring tetracycline resistance and fusaric acid sensitivity, a β-galactosidase gene (lacZ) conferring the ability to catabolize 5-bromo-4-chloro-3-indolyl-β-D-galactopyranoside (X-Gal, which facilitates blue/white colony screening), or a lactose permease gene (lacY, which facilitates transportation of X-Gal into the engineered bacterium). The inset panel depicts 50 bp on either side of a scarless mutation (e.g., deletion) that can be produced by the vector. The inset panel depicts elimination of the whole bcsABCD operon, beginning at the bcsA start codon and ending at the bcsD stop codon, from the target genome.

FIG. 2B shows an expanded view of a gene-modification vector that can be used to make an engineered bacterium that comprises a truncation of wcaE, thus ablating WcaE activity in the engineered bacterium. The vector comprises at least upstream and downstream homologous nucleotide sequences (700-800 bp each) corresponding to the gene or sequence of interest targeted for mutation. In this vector, the genetic modification sequence (e.g., “ΔwcaE truncated”) is comprised in the homologous nucleotide sequences. The upstream homologous nucleotide sequence (e.g., “Upstream Homology”) comprises homology to an ebsC coding sequence, is upstream of the gene of interest targeted for truncation (e.g., wcaE), and comprises the genetic modification sequence. The downstream homologous nucleotide sequence (e.g., “Downstream Homology”) comprises homology to a mutT coding sequence and is downstream of the gene of interest targeted for truncation (e.g., wcaE). Together, the upstream and downstream homologous nucleotide sequences comprise a truncated wcaE sequence that replaces a native wcaE sequence in a target genome. The vector may also comprise an aminoglycoside phosphotransferase gene (aphA1) conferring kanamycin resistance, a levansucrase gene (sacB) conferring sucrose sensitivity, a class A tetracycline resistance protein (tetA) gene conferring tetracycline resistance and fusaric acid sensitivity, a β-galactosidase gene (lacZ) conferring the ability to catabolize 5-bromo-4-chloro-3-indolyl-β-D-galactopyranoside (X-Gal, which facilitates blue/white colony screening), or a lactose permease gene (lacY, which facilitates transportation of X-Gal into the engineered bacterium). The inset panel depicts 50 bp on either side of the scarless mutation that can be produced by the vector (e.g., partial wcaE deletion), where the wcaE sequence between the 15th codon and stop codon are eliminated from the chromosome. The inset panel depicts a sequence that results in synthesis of only a 15 amino acid non-functional N-terminal fragment of wild type WcaE.

FIG. 2C shows an expanded view of a gene-modification vector that can be used to make an engineered bacterium that comprises an insertion that leads to constitutive expression of gqqA. The vector comprises at least upstream and downstream homologous nucleotide sequences (700-800 bp each) corresponding to the gene or sequence of interest targeted for mutation and a genetic modification sequence (e.g., “pTac1 constitutive promoter” and “optimized RBS”) flanked by the upstream and downstream homologous nucleotide sequences. The upstream homologous nucleotide sequence (e.g., “Upstream Homology”) comprises homology to an kdsB coding sequence and is upstream of the gene of interest targeted for enhanced expression (e.g., gqqA). The downstream homologous nucleotide sequence (e.g., “Downstream Homology”) comprises homology to a gqqA (pheA2) coding sequence, which is the targeted for enhanced expression (e.g., gqqA), and is downstream of the sequence of interest targeted for insertion (e.g., pTac1 constitutive promoter and optimized RBS). The vector may also comprise an aminoglycoside phosphotransferase gene (aphA1) conferring kanamycin resistance, a levansucrase gene (sacB) conferring sucrose sensitivity, a class A tetracycline resistance protein (tetA) gene conferring tetracycline resistance and fusaric acid sensitivity, a β-galactosidase gene (lacZ) conferring the ability to catabolize 5-bromo-4-chloro-3-indolyl-β-D-galactopyranoside (X-Gal, which facilitates blue/white colony screening), or a lactose permease gene (lacY, which facilitates transportation of X-Gal into the engineered bacterium). The inset panel depicts 30 bp on either side of the inserted tac promoter and synthetic RBS made by the vector. The inset panel depicts a sequence that results in expression the gqqA gene subject to control by the promoter and RBS elements inserted upstream of the gqqA coding sequence in the target genome.

### DETAILED DESCRIPTION

Scaled production of industrially relevant bioproducts can be costly in light of bacterial production limitations. First, bacterial production may be limited by reduced bioavailability of cellular resources and downstream processing steps associated with cellulose produced by the bacterium in addition to the bioproduct. Second, bacterial production may be limited by physical and chemical foam mitigation treatment steps associated with foam produced by the bacterium in addition to the bioproduct. Third, bacterial production may be limited by volumetric productivity of the bacterium during fermentation. Thus, engineering a bacterium to overcome these limitations may improve bacterial production of industrially relevant bioproducts by reducing cellulose production, reducing foam formation, and improving cell density (e.g., improving volumetric productivity) during fermentation.

Provided herein, in some embodiments, are compositions and methods for enhancing bioprocessing and culture handling outcomes associated with fermentation-based production of industrially relevant bioproducts. The engineered bacterium of the compositions and methods provided herein comprise a reduced capacity for cellulose formation, a reduced capacity for foaming behavior, and/or an increased capacity for cell density compared to a wild type bacterium. These improvements may reduce costs associated with removing cellulose and mitigating foam formation. These improvements may improve volume to product ratios in bioreactors. Fermentative production of bioproducts using the engineered bacterium of the compositions and methods provided herein may improve bioproduct yields compared to fermentative production of the bioproduct using a wild type bacterium.

### Engineered Bacterium

Disclosed here, in some embodiments, is an engineered bacterium comprising: (a) a reduced capacity for cellulose formation compared to a capacity for cellulose formation of a wild type acetic acid bacterium (AAB), (b) a reduced capacity for foaming behavior compared to a foaming behavior of said wild type AAB, (c) an increased capacity for cell density compared to a capacity for cell density of said wild type AAB, or (d) a combination thereof.

The engineered bacterium may comprise a modification of: (a) at least one gene that inhibits cellulose formation via gene disruption or downregulation, (b) at least one gene that enhances cellulose degradation via gene upregulation, (c) at least one gene that depresses foaming behavior via gene disruption or downregulation, (d) at least one gene that depresses foaming behavior via gene upregulation, (e) at least one gene that reduces cell density via gene disruption or downregulation.

The engineered bacterium may comprise a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; (d) at least one gene selected from the group consisting of: a gene encoding N-Acyl-homoserine lactone acylase GqqA and a gene encoding N-Acyl-homoserine lactone lactonase Qsdr1; (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein; or (f) a combination thereof.

The engineered bacterium may be a Gram-negative bacteria. In some instances, the engineered bacterium may be within the family Acetobacteraceae (e.g., an acetic acid bacterium (AAB)). Acetic acid bacteria are widespread and versatile organisms that produce numerous natural and industrially valuable products. The engineered bacterium may be of a species from the genera Acetobacter, Gluconacetobacter, Gluconobacter, or Komagataeibacter. In some instances, the engineered bacterium is from the genus Komagataeibacter. In some instances, the engineered bacterium is from the species Komagataeibacter europaeus. In some instances, the engineered bacterium may be Komagataeibacter europaeus LMG 1521. The engineered bacterium may be capable of oxidizing ethanol to produce acetic acid.

The engineered bacterium may comprise any combination of the modifications described herein. In some instances, the engineered bacterium may comprise a modification of at least one gene. The modification may be accomplished via homologous recombination, a transposase-based system, a CRISPR-Cas system, an integrase-based system, a recombinase-based system, a viral vector, a TALEN (Transcription Activator-Like Effector Nuclease)-based system, a Zinc Finger Nuclease (ZFN)-based system, a lambda Red system, site-directed mutagenesis, or other suitable methods.

In some instances, the at least one gene is a natively expressed gene and the modification comprises knockout, disruption, truncation, knockdown, or inhibition of the at least one gene. In some instances, the at least one gene is a natively expressed gene and the modification results in increased expression of the at least one gene. In some instances, the at least one gene is a natively expressed gene and the modification results in the increased activity of a product corresponding to the gene of interest. In some instances, the at least one gene is a non-natively expressed gene and the modification comprises heterologous expression of the at least one gene. In some instances, the modification leads to increased activity of a gene product (e.g., protein) associated with the at least one gene.

### Cellulose Formation

Bacterial cellulose (BC) is an extracellular polymer produced by some bacterium. The production of BC can inhibit the bacterium from synthesizing other bioproducts. BC production can inhibit synthesis of other bioproducts by acting as a competing carbon sink or a competing energy sink during the utilization of finite cellular resources. BC production can inhibit synthesis of other bioproducts by leading to the production of cellulose aggregates. Cellulose aggregates may inhibit uniform distribution of a plurality of bacteria within a liquid fermentation media, impeding ideal fermentation conditions. Cellulose aggregates may impede downstream processing of the fermentation broth (produced by fermenting the bacterium in the liquid media) to obtain a desired bioproduct produced by the bacterium.

In some instances, the engineered bacterium comprises a reduced capacity for cellulose formation compared to a capacity for cellulose formation of a wild type AAB. This reduced capacity for cellulose formation may comprise no cellulose formation. The elimination or reduction of cellulose production in the engineered bacterium may mitigate or eliminate a competing pathway for metabolic resources in the bacterium. The elimination or reduction of cellulose production in the engineered bacterium may improve bacterial distribution within a liquid fermentation media. Improved bacterial distribution in turn improves substrate utilization. Improved bacterial distribution also improves product synthesis kinetics in the fermentation media. The elimination or reduction of cellulose production in the engineered bacterium may reduce the complexity associated with removing cellulose as part of downstream processing of the fermentation broth.

In some instances, the engineered bacterium comprises a modification of at least one gene to reduce cellulose formation. Non-limiting examples of genes that can be modified to reduce cellulose formation include a gene encoding a bacterial cellulose synthesis protein, a gene encoding phosphoglucomutase, a gene encoding a DTP-glucose-1-phosphate uridylyltransferase, a gene encoding a diguanylate cyclase, a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1.

In some instances, the engineered bacterium comprises a modification of at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase. In some instances, the modification comprises knockout, disruption, truncation, knockdown, or inhibition of the at least one gene.

In some instances, the engineered bacterium comprises a modification of at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1. In some instances, the modification comprises enhancing expression of, heterologously expressing, or enhancing activity of a gene product (e.g., protein) associated with the at least one gene. In some instances, the modification comprises heterologous expression of at least one gene selected from the group consisting of: a gene encoding a soluble cellulase protein, a gene encoding endoglucanase CelY, a gene encoding endoglucanase CelZ, and a gene encoding N-Acyl-homoserine lactone lactonase Qsdr1. In some instances, the modification comprises enhancing expression of a gene encoding N-Acyl-homoserine lactone acylase GqqA.

Modification of the genes that encode the bacterial cellulose synthesis ABCD proteins (e.g., ΔbcsABCD) (e.g., bacterial cellulose synthase catalytic subunit A (SEQ ID NO: 1), bacterial cellulose synthase subunit B (SEQ ID NO: 2), bacterial cellulose synthase subunit C (SEQ ID NO: 3), and bacterial cellulose synthase subunit D (SEQ ID NO: 4), respectively) may reduce cellulose formation.

Modification of a gene that encodes Phosphoglucomutase (e.g., Δpgm) may reduce cellulose formation. Pgm (SEQ ID NO: 5) catalyzes the first committal step toward biosynthesis of the substrate used for bacterial cellulose production.

Modification of a gene that encodes DTP-glucose-1-phosphate uridylyltransferase (e.g., ΔgalU) may reduce cellulose formation. GalU (SEQ ID NO: 6) catalyzes the production of UDP-Glucose, the substrate for bacterial cellulose production.

Modification of a gene that encodes diguanylate cyclase may reduce cellulose formation. Diguanylate cyclase catalyzes the formation of Bis-(3′-5′)-cyclic dimeric guanosine monophosphate (c-di-GMP). c-di-GMP activates cellulose production. Therefore, reduction in c-di-GMP may decrease gene expression of bcsABCD and therefore cellulose production.

Heterologous expression of a soluble cellulase protein may also reduce overall cellulose formation by enhancing degradation of any cellulose produced. In some instances, the engineered bacterium is added as a microbial addition to a mixed culture (e.g., microbial community) of bacterial cellulose-producing bacteria. Heterologous expression of the soluble cellulase protein by the engineered bacterium may reduce cellulose contributed by other bacteria in the mixed culture. Heterologous expression of endoglucanase CelY (SEQ ID NO: 7) or endoglucanase CelZ (SEQ ID NO: 8) from the plant saprophyte Dickeya dadantii 3937 may reduce overall cellulose formation. Heterologous expression of a non-native gene may be carried out through integration of the non-native gene into the engineered bacterium or through expression from a replicative plasmid delivered to the engineered bacterium.

Expression of N-Acyl-homoserine lactone acylase GqqA or N-Acyl-homoserine lactone lactonase Qsdr1 may disrupt cellulose aggregate formation. In some instances, native N-Acyl-homoserine lactone acylase (GqqA (SEQ ID NO: 9)) expression may be enhanced. In some instances, recombinant N-Acyl-homoserine lactone lactonase Qsdr1 (SEQ ID NO: 10) from Sinorhizobium fredii NGR234 may be heterologously expressed. Expression of N-Acyl-homoserine lactone acylase GqqA or N-Acyl-homoserine lactone lactonase Qsdr1 may reduce cellulose contributed by other bacteria in a mixed culture.

TABLE 1A
Illustrative Proteins Relevant To Reducing Cellulose Formation

| SEQ ID | | |
|---|---|---|
| NO: | Identifier | Amino Acid Sequence |
| 1 | BcsA | MSEVQSSAPAESWFGRFSNKILSLRGASYIVGAVGLFALLAATIVILSIHEQLIVALI |
| | | CVAVFFIVGRRKSRRTQVFLEVLSALVSLRYLTWRLTETLDFDTWIQGLLGVTLLL |
| | | AELYALYMLFLSYFQTISPLHRAPLPLSPNPDEWPTVDIFIPTYDEDLSIVRLTVLAA |
| | | LGIDWPPEKMNVYILDDGKRETFGRFAEECGARYITRPDNTHYKAGNLNHAIKQT |
| | | RGDFILILDCDHIPTRAFLQIAMGWMVEDPKIALMQTPHHFYSPDPFQRNLAVGYR |
| | | TPPEGNLFYGVIQDGNDFWDATFFCGSCAILRRKAIEEIGGFATETVTEDAHTALR |
| | | MQRKGWSTAYLRIPLASQLATERLITHIGQRMRWARGMIQIFRVDNPMLGPGLKL |
| | | GQRLCYLSAMTSFFFAIPRVIFLASPLAFLFFNQNIIAASPLAVLAYAIPHMFHSIATA |
| | | AKVNRGWRYSFWSEVYETVMALFLVRVTIVTMIFPSKGKFNVTEKGGVLENETFD |
| | | LDATYPNIIFAVVMALGLLRGAYALIFQHLDIIAERAYALNCIWSVISLIILLAAIAV |
| | | GRETKQIRHSHRIEAHIPVTVYDYEGNSSHGITEDVSMGGVAIHMPWRDVTPDQPV |
| | | QVVVHAILEGEEVNLPATMIRSANGKAVFTWSINNIQIEASVVRFVFGRADAWLQ |
| | | WNNYEDDRPLRSLWSLILSIKALFRKKGQMIAHSRPKKKPIALPVERREPTTSQGG |
| | | QKQEGKISRAAS* |
| 2 | BcsB | MRPRDMKMVSLIALLVFATGAQAAPIASKAPVPQPTGNDLPPLPAAAPAAAQPQA |
| | | QAGNNAAPAAAPAGDAGSNADAILDNAENATGASSDVATVHTYSLQELGAAGAL |
| | | TMRGTAPLQGLQFGIPADQLVTSARLVVSGAMSPNLQPDNSTVTITLNEQYIGTLR |
| | | PDPSHPAFGPLSFDVNPIFFVSGNRLNFSFATGSKGCTDPSNGLQWASVSEHSQLQI |
| | | TTIPLPPRRQLARLPQPFFDKTVRQKVVIPFVLAQTFDPEVLKASGIIASWFGQQTD |
| | | YRGVNFPVFSTIPQTGNAIVVGVADELPSALGRPSVSGPTLMEVANPSDPNGTLLL |
| | | VTGRDRDEVITASKGIGFGSSALPVASRMDVAPIDVAPRVANDAPSFLPTTRPVRL |
| | | GELVPVSALQGEGYTPGVLSVPFRVSPDLYTWRDRPYKLNVRFRAPDGPILDVARS |
| | | HLDVGINNTYLQSYSLREQNSVVDQLLRRVGVGTQNAGVEQHTLTIPPWMVFGQ |
| | | DQLQFYFDAAPLTEPGCRPGPSLIHMSVDPDSTIDLSNAYHITRMPNLAYMASAGY |
| | | PFITYADLSRSAVVLPDHPNGTVVSAYLDLMGFMGATTWYPVSGVDIVSADHVS |
| | | DVADRNLIVLSTLSNSGEVSSLLSNSAYQISDGRLHMGLRSTLSGVWNIFQDPMSVI |
| | | SNTHPTEVETTLTGGVGAMVEAESPLASGRTVLALLSGDGQGLDNLVQILGQRKN |
| | | QAKVQGDLVLAHGDDLTSYRSSPLYTVGTLPMWLMPDWYMHNHPIRVVVVGLF |
| | | GCLLVVAVLVRALFRHAMYRRRQLQEERQKS* |
| 3 | BcsC | MIMSRRYVFSLSAGLLASSCMGVMMAVPVARAQQASTAMTGAQATGGTAAPRQI |
| | | LLQQARFWLQQQQYDNARQALQNAQRIAPDSPDVLEVQGEYQTAIGNREAAADT |
| | | LRHLQQVAPGSTAANSLTDLLHERSISTGDLSQVRALAASGHNEQAVAGYQKLFN |
| | | GGKPPHSLAVEYYQTMAGVPADWDQARAGLASVVAANPQDYRAQLAFAQALTY |
| | | NTPTRMEGLARLKDLEGFRAQAPVEAAAAAQSYRQTLSWLPVTPETQPLMQQWL |
| | | AAHPNDTALKEHMLHPPGGPPDKAGLARQAGYQQLNAGRLSAAEQSFQSALQIN |
| | | AHDADSLGGMGLVSMRQGDAAEARRYFQEAMAADPKTADRWRPALAGMEISG |
| | | DYAAVRQMIAAHQYTEAKQRLTSLARQPGQFTGATLMLADLQRTTGQDASEQS |
| | | YRSVLARDPNSQLALMGLARVDMAQGNTAEARQLLSRVGPQYAEQVGEIEVTGL |
| | | MAAASHTSDSARKVSILREAMTQAPRDPWVRINLANALQQQGDVAEAGRVMQPI |
| | | LANPVTAQDRQAGILYTYGAGNDAATRRLLSGLSPEDYSPAIRSIATEMEIKEDLAS |
| | | RLSMVANPVPLIREALSPPDPTGARGVAVADLFRQRGDMLHARMALRIASTRTIDL |
| | | SPDQRLAYATEYMKISNPVAAARLLAPLGDGSGSGMGNALPPEQQQTLQQLRMGI |
| | | SVAQSDLLNQRGDQAQAYDQLAPALRADPEATAPKLALARLYNGEGKASKALEI |
| | | DLAVLRHNPQDLDARQAAVQAAVNSGHKSLATRLAMDGVQESPMDARAWLSM |
| | | AVADQADGHGHRTIADLRRAYDLRLQQVEGSRMAAGAGAAGAEQDALAPPSSN |
| | | PFRHHGYGRQTELGAPVTGGSYSMEATSPEASDQMLSSISGQINTLRENLAPSIDGG |
| | | LGFRSRSGEHGMGRLTEANIPIVGRLPLQAGESSLTFSITPTMIWSGDLDSGSVYDV |
| | | PRFGTDMATQAWNQYVNYMNRNNQGSTSRYKSQLVKGGEGEAGFAPDVQFGNS |
| | | WVRADVGASPIGFPITNVLGGVEFSPRVGPVTFRVSAERRSITNSVLSYGGMRDPN |
| | | YYSAIGRYARSLYGKELASQWSQEWGGVVTNHFHGQVEATLGNTIVYGGGGYAI |
| | | QTGKNVRRNSEREAGIGVNTLVWHNANMLVRIGVSLTYFGYGNNQDFYTYGQG |
| | | GYFSPQSYYAATYPIRYAGQHKRLDWDVNASYGYQVFHEHSSPFFPTSSLLQSGA |
| | | QYVANNYVTNATSSDYLSQETVNSAYYPGDSVASLTGGFNARVGYRFTHNLRLD |
| | | LSGRWQKAGNWTESGAMISAHYLIMDQ* |
| 4 | BcsD | MTTFNAKPDFSLFLQALSWEIDDQAGIEVRNDLLREVGRGMAGRLQPPLCNTIHQL |
| | | QIELNALLGMINWGYVKLELLAEEQAMRIVHEDLPQVGSAGEPSGTWLAPVLEGL |
| | | YGRWITSQPGAFGDYVVSRDVDAEDLNSVPTQTVILYMRTRSNSN* |
| 5 | Pgm | MIFRHRFDPTSLREYDIRGTVGKTLGPEDAYAIGRTFASVVAGDGGRTVVVGYDG |
| | | RLSSPALERALVEGAMASGMDVVRIGCGPTPMLYFASADLNADGAVMVTGSHNP |
| | | PDQNGFKIVLANRPFFGAQIAMLGQMAATQNVVAQASGTTRMVDIRAAYIDRLLR |
| | | DHDHDARALNVVWDCGNGAAGDVLSLLVKRLPGRHRVLNAAIDGRFPAHHPDP |
| | | TIPANLQQLITAVRQDGADLGIAFDGDADRLGVVDDTGAIVWADQLLLILARDML |
| | | RARPGATIIADIKTSQVVFDEIDRAGGRALMWKSGHSQMKERMAETGALLAGEMS |
| | | GHLFFADRWYGFDDALYAALRLLDVISRLDGSLSDARRALPATVSTPELRFACPDT |
| | | RKFDVITEVATRLAQAGADVCDIDGVRVSTPDGWWLLRASNTQAVLVARAEGAT |
| | | QAALDRLKAALSAQLAQSGVTLPDHADPATPH* |
| 6 | GalU | MIKPLKKAVLPVAGLGTRFLPATKCVPKEMLTVVDRPLIQYAIDEAREAGIEEFCL |
| | | VSSRGKDSLIDYFDISYELEDTLKARKKTSALKALEATRVIPGTMLSVRQQEPLGLG |
| | | HAIWCAREFIGNDPFAILLPDDVVQSKKSCIGQLVEVYNKTGGNVLAVTEVPREQT |
| | | GSYGILDVGKDDGKTVEVKGLVEKPDPKDAPSTLSVIGRYVLTADVLKHLAKLEK |
| | | GAGGEVQLTDAMAKTIGHVPFHGYRYEGKRFDCGSKIGFLEAQIAFALEREELAP |
| | | QVREFLTKYK* |
| 7 | CelY | MGKPMWRCWALMLMVWFSASATAANGWEIYKSRFMTTDGRIQDTGNKNVSHT |
| | | EGQGFAMLMAVHYDDRIAFDNLWNWTQSHLRNTTSGLFYWRYDPSAANPVVDK |
| | | NNASDGDVLIAWALLKAGNKWQDNRYLQASDSIQKAIIASNIIQFAGRTVMLPGA |
| | | YGFNKNSYVILNPSYFLFPAWRDFANRSHLQVWRQLIDDSLSLVGEMRFGQVGLP |
| | | TDWAALNADGSMAPATAWPSRFSYDAIRIPLYLYWYDAKTTALVPFQLYWRNYP |
| | | RLTTPAWVDVLSSNTATYNMQGGLLAVRDLIMGNLDGLSDLPGASEDYYSSSLR |
| | | LLVMLARGK* |
| 8 | CelZ | MPLSYLDKNPVIDSKKHALRKKLFLSCAYFGLSLACLSSNAWASVEPLSVNGNKIY |
| | | AGEKAKSFAGNSLFWSNNGWGGEKFYTADTVASLKKDWKSSIVRAAMGVQESG |
| | | GYLQDPAGNKAKVERVVDAAIANDMYAIIGWHSHSAENNRSEAIRFFQEMARKY |
| | | GNKPNVIYEIYNEPLQVSWSNTIKPYAEAVISAIRAIDPDNLIIVGTPSWSQNVDEAS |
| | | RDPINAKNIAYTLHFYAGTHGESLRNKARQALNNGIALFVTEWGTVNADGNGGV |
| | | NQTETDAWVTFMRDNNISNANWALNDKNEGASTYYPDSKNLTESGKKVKSIIQS |
| | | WPYKAGSAASATTDPSTDTTTDTTVDEPTTTDTPATADCANANVYPNWVSKDWA |
| | | GGQPTHNEAGQSIVYKGNLYTANWYTASVPGSDSSWTQVGSCN* |
| 9 | GqqA | MNGERIIAFQGRPGAYSDLACRQARPQWTTLPCQTFAQTIAAVHDGRAELAMLAC |
| | | ENSLAGRVPDIHALLPEAGLFIVGEHFQRVEHCLLGIPGSTLADARRIHTHPVAMA |
| | | QVRGIITELGLDPVVEFDTAGAAEMVREWGRKEDVAVASALAAELNGLEILRRNV |
| | | EDATHNTTRFYIASRRPATLPPPGPGFMTTLLFRVNNQPGALYKALGGLATAGVN |
| | | MTRLESYMLEGSFSATQFLMDVEGHPEAPPLARALDELSFFSEQQEILGVYPASPFR |
| | | RKP* |
| 10 | QsdR1 | MPHAETNRRTGQQETTMSLDNASRPSRSGRDELVPSRYAVKVGEIDVLVISDGVLP |
| | | IPTPVLATNADPAVRTAWLDDMFLPTDVLHWPLNVVLVRSGGQAILVDAGLGVEF |
| | | PDFPRAGQTVSRLEAAGVDLASVTDVVLTHMHMDHIGGLLADGVKDRLRPDLRI |
| | | HVAAAEVKFWEAPDFSHASMPSTVPPVLRRTAKRFMAEYQSQLRQFDEDYEVAP |
| | | GVVVTRTGGHTPGHSVVRLASGGDRLTFAGDAVFQVGFDHPDWHNGFEHDPEEA |
| | | ARVRVRLLRELAATREPLVATHLPFPSVCHVAADGDVFRWVPVVWDS* |

n some instances, a gene encoding a protein having an amino acid sequence with at least 70%, 75%, 80%, 85%, 90%, 95%, or 100% sequence identity to any one of the amino acid sequences in Table 1A is modified.

n some instances, the at least one gene encodes a protein with at least 70%, 75%, 80%, 85%, 90%, 95%, or 100% sequence identity to SEQ ID NO: 1. In some instances, the at least one gene encodes a protein with at least 90% sequence identity to SEQ ID NO: 1. In some instances, the at least one gene encodes a protein with at least 70%, 75%, 80%, 85%, 90%, 95%, or 100% sequence identity to SEQ ID NO: 2. In some instances, the at least one gene encodes a protein with at least 90% sequence identity to SEQ ID NO: 2. In some instances, the at least one gene encodes a protein with at least 70%, 75%, 80%, 85%, 90%, 95%, or 100% sequence identity to SEQ ID NO: 3. In some instances, the at least one gene encodes a protein with at least 90% sequence identity to SEQ ID NO: 3. In some instances, the at least one gene encodes a protein with at least 70%, 75%, 80%, 85%, 90%, 95%, or 100% sequence identity to SEQ ID NO: 4. In some instances, the at least one gene encodes a protein with at least 90% sequence identity to SEQ ID NO: 4. In some instances, the at least one gene encodes a protein with at least 70%, 75%, 80%, 85%, 90%, 95%, or 100% sequence identity to SEQ ID NO: 5. In some instances, the at least one gene encodes a protein with at least 90% sequence identity to SEQ ID NO: 5. In some instances, the at least one gene encodes a protein with at least 70%, 75%, 80%, 85%, 90%, 95%, or 100% sequence identity to SEQ ID NO: 6. In some instances, the at least one gene encodes a protein with at least 90% sequence identity to SEQ ID NO: 6. In some instances, the at least one gene encodes a protein with at least 70%, 75%, 80%, 85%, 90%, 95%, or 100% sequence identity to SEQ ID NO: 7. In some instances, the at least one gene encodes a protein with at least 90% sequence identity to SEQ ID NO: 7. In some instances, the at least one gene encodes a protein with at least 70%, 75%, 80%, 85%, 90%, 95%, or 100% sequence identity to SEQ ID NO: 8. In some instances, the at least one gene encodes a protein with at least 90% sequence identity to SEQ ID NO: 8. In some instances, the at least one gene encodes a protein with at least 70%, 75%, 80%, 85%, 90%, 95%, or 100% sequence identity to SEQ ID NO: 9. In some instances, the at least one gene encodes a protein with at least 90% sequence identity to SEQ ID NO: 9. In some instances, the at least one gene encodes a protein with at least 70%, 75%, 80%, 85%, 90%, 95%, or 100% sequence identity to SEQ ID NO: 10. In some instances, the at least one gene encodes a protein with at least 90% sequence identity to SEQ ID NO: 10.

In some instances, the engineered bacterium comprises a reduced capacity for cellulose formation compared to a capacity for cellulose formation by a wild type AAB. In some instances, the engineered bacterium comprises a reduced capacity for cellulose formation by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to a capacity for cellulose formation of a reference bacterium (e.g., a wild type AAB or an AAB variant that does not comprise the modification described herein). In some instances, the engineered bacterium may comprise a reduced capacity for cellulose formation by at least about 5% to about I 00% compared to a capacity for cellulose formation of by a wild type AAB, or any range defined therein.

In some instances, the capacities for cellulose formation are measurable by detection of cellulose produced by bacteria in a cell culture vessel (e.g., flask, bioreactor). A capacity for cellulose formation may be measurable by detection of cellulose produced in liquid samples of the cell culture. The samples may be treated with cellulase, and then glucose produced by the cellulase treatment may be quantitatively measured via high-performance liquid chromatography (HPLC). A glucose measurement may correlate to a capacity for cellulose production. The absence of cellulose formation in the engineered bacterium may also be assessed as the qualitative loss of cellulose aggregate accumulation in the cell culture vessel, as compared to aggregates observed in a culture containing a reference bacterium grown in the absence of cellulase.

### Foaming Behavior

Foam may be generated during fermentation of bacteria. If left untreated, foam formation may lead to both culture broth losses and bioproduct losses. Traditionally, physical and chemical means of foam mitigation may be employed during industrial fermentations. However, physical and chemical foam treatment can raise fermentation-associated costs. Genetic engineering to reduce foam formation circumvents reliance upon these cost-added foam treatment technologies.

In some instances, the engineered bacterium comprises a reduced capacity for foaming behavior compared to a foaming behavior of a wild type AAB. In some instances, the engineered bacterium may produce less foam during fermentation than a wild type AAB. In some instances, the engineered bacterium comprises a reduced capacity for expression of GinA compared to a capacity for expression of GinA of a wild type AAB.

In some instances, the engineered bacterium comprises a modification of at least one gene to reduce foaming behavior or foam formation. Non-limiting examples of genes that can be modified to reduce foaming behavior include a gene encoding a GT2 family Glycosyltransferase, a gene encoding a N-Acyl-homoserine lactone-dependent transcriptional regulator, a gene encoding an Acyl-homoserine-lactone synthase, a gene encoding an Acyl-homoserine-lactone response regulator, a gene encoding N-Acyl-homoserine lactone acylase GqqA, and a gene encoding N-Acyl-homoserine lactone lactonase Qsdr1.

In some instances, the engineered bacterium comprises a modification of at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator. In some instances, the modification comprises knockout, disruption, truncation, knockdown, or inhibition of the at least one gene.

In some instances, the engineered bacterium comprises a modification of a gene encoding N-Acyl-homoserine lactone acylase GqqA or a gene encoding N-Acyl-homoserine lactone lactonase Qsdr1. In some instances, the modification comprises enhancing expression of, heterologously expressing, or enhancing activity of a gene product (e.g., protein) associated with the at least one gene.

Modification of a gene encoding GT2 family Glycosyltransferase (e.g., ilwcaE) may reduce foam formation. WcaE (SEQ ID NO: 11) plays a role in foam formation.

Modification of N-Acyl-homoserine lactone acylase GqqA or N-Acyl-homoserine lactone lactonase Qsdr1 can depress activation of GinA (SEQ ID NO: 14)-dependent antifoaming repression. Many Gram-negative bacteria use N-acylhomoserine lactone (AHL)-dependent quorum-sensing systems to regulate gene expression. Expression of N-Acyl-homoserine lactone acylase GqqA or N-Acyl-homoserine lactone lactonase Qsdr1 may depress activation of GinA-dependent antifoaming repression via degradation of a N-Acyl-homoserine quorum-sensing signal molecule that activates GinA expression. In some instances, native N-Acyl-homoserine lactone acylase (GqqA (SEQ ID NO: 9)) expression may be enhanced. In some instances, recombinant N-Acyl-homoserine lactone lactonase Qsdr1 (SEQ ID NO: 10) from Sinorhizobium fredii NGR234 may be heterologously expressed. In some instances, the engineered bacterium is added as a microbial addition to a mixed culture (e.g., microbial community) of foam-producing bacteria. Increased activity of N-Acyl-homoserine lactone acylase GqqA or N-Acyl-homoserine lactone lactonase Qsdr1 may reduce foam formed by other bacteria in the mixed culture.

Modification of a gene that encodes N-Acyl-homoserine lactone-dependent transcriptional regulator (e.g., ΔginR) may reduce foam formation. GinR (SEQ ID NO: 12) may decrease expression of GinA (SEQ ID NO: 14), consequently leading to reduced GinA-dependent anti-foaming repression. [0090] Modification of a gene that encodes Acyl-homoserine-lactone synthase (e.g., Δginl) may reduce foam formation. GinI (SEQ ID NO: 13) may decrease expression of GinA, consequently leading to reduced GinA-dependent anti-foaming repression.

Modification of a gene that encodes Acyl-homoserine-lactone response regulator (e.g., ΔginA) may reduce foam formation. GinA (SEQ ID NO: 14) mitigation may lead to reduced GinA-dependent antifoaming repression.

TABLE 1B
Illustrative Proteins Relevant To Reducing Foam Formation

| SEQ ID | | |
|---|---|---|
| NO: | Identifier | Amino Acid Sequence |
| 9 | GqqA | MNGERIIAFQGRPGAYSDLACRQARPGWTTLPCQTFAQTIAAVHDGRAELAMLAC |
| | | ENSLAGRVPDIHALLPEAGLFIVGEHFQRVEHCLLGIPGSTLADARRIHTHPVAMA |
| | | QVRGIITELGLDPVVEFDTAGAAEMVREWGRKEDVAVASALAAELNGLEILRRNV |
| | | EDATHNTTRFYIASRRPATLPPPGPGFMTTLLFRVNNQPGALYKALGGLATAGVN |
| | | MTRLESYMLEGSFSATQFLMDVEGHPEAPPLARALDELSFFSEQQEILGVYPASPFR |
| | | RKP* |
| 10 | QsdR1 | MPHAETNRRTGQQETTMSLDNASRPSRSGRDELVPSRYAVKVGEIDVLVISDGVLP |
| | | IPTPVLATNADPAVRTAWLDDMFLPTDVLHWPLNVVLVRSGGQAILVDAGLGVEF |
| | | PDFPRAGQTVSRLEAAGVDLASVTDVVLTHMHMDHIGGLLADGVKDRLRPDLRI |
| | | HVAAAEVKFWEAPDFSHASMPSTVPPVLRRTAKRFMAEYQSQLRQFDEDYEVAP |
| | | GVVVTRTGGHTPGHSVVRLASGGDRLTFAGDAVFQVGFDHPDWHNGFEHDPEEA |
| | | ARVRVRLLRELAATREPLVATHLPFPSVCHVAADGDVFRWVPVVWDS* |
| 11 | WcaE | MAVSSNNAEMADDWATFDPAWYRARYAAMLDLMDIPVEQTREFHDAHGAALG |
| | | HAPNPFFDEEWYRATYPDVAAQVTAGTWRSGFDHYLNAGARTHNPHWLFDERA |
| | | YLAACPDITPATLDAGGYRNAYDHYLRVGDGEMRGGSCFFDPETCIALLAECDQD |
| | | GTRRPFAAYLRRGMDALPCRSVSLYFDAEWYVQAYPEAHAEITQGLWRSALHHY |
| | | LCNPTPQAFDPGPFFSESFYAMVNPDVQAAIEAGALRNGYAHFLSDGVHEQRKPC |
| | | STLDLAHYMRDPGVQADIAGRRARDGLGHYLAARPDLKPPPPPQVTEDQARALFR |
| | | HMCAARLPLLLDGRIDFTTDAPPALSVIIVAHDQFELTMSTLASLRANYHGPMQVV |
| | | VVDSGSRDGVAGIEDHVRGIEVLRFAGNIGFVRGCNAALRRVRAPATLYLNNDVD |
| | | LQYGAIANALSRLMADETTGAVGGRIIRTHGMLQEAGSMIWRDGSVQGYMRDAH |
| | | PCVPEAGFVRAVDFCSGVFLMVRTEVAQVLDGFDESYAPAYFEETDLCVRIRALG |
| | | YRILYDPAVCLVHYECGTSDGTSASRLIARNSDLFIRRHASGLRRRLLRHAPNEAR |
| | | ARHADDGRHILFIEDRLPLRHLGSGFTRSHDIVTTLAGLGYHVTIYPIFRPIEDAATR |
| | | AAAFPENVEVIHDRELPDLPAFLRARSGCFDAIWIARTHNAARVASILNDAASYIPA |
| | | GRIVVDTEALAACRDMEYDRLHAITPSRPLDARLADELRPLFVAQRVVAVNTAEA |
| | | DLLRGAGFDNVSVLGHVQAPRATGPGWAARRDILFLGAVHDMASPNLDSLAWFS |
| | | GQVLPLLVAQLGPDIRFSVCGHASPRVDLGPLRHHPNVRMLGRVADTAPVYDHH |
| | | RVFVAPTRYGAGIAYKLHEAAANGLPVVGSHLLCRQAGWRDGQDMLCASITDPA |
| | | DFARQVVRLYHDQVLWDTVRDNALTRITTEHAPQDYRNAVATIMDAVFTRG* |
| 12 | GinR | MVGWTIDLLAKLHDLENAATKHDLLSIYLDAVLSVGNVHVTIVELNRIEDPKENFI |
| | | HVGYPTEWVNFYIENNYIVSDPIIKKSRFMSHPYFWHEIRNINKSEKKIIRDVSEFGI |
| | | KKGLTIPVHTHDRLIYAICFAFTDKNVDQEIELYLRALSNFFIAGYKKLDEPTDMSP |
| | | PILTPREKECLRWTAKGKSSWETGMIVGVSERTVNFHINNALLKLKCTNRIMGVV |
| | | RAICAGLIEL* |
| 13 | GinI | MNDPLIQQEESGSMIEVVTVENAHWCGTALAEQFKFRYRHFVANEQWEVPFYKG |
| | | MEYDQFDTPAAVYLVWRDVAGVVRGMIRLLPTNRPYMLETLWPDMMPDPIVLP |
| | | GPTVWEITRFGVERNLSLSLRKQISAELILACIEFSVLNDIHTYLFLTAWGVLKRIVP |
| | | GAGVDAQIHSRKTLPSGHDVASAVVPVSQAVLDKARAKLNIHYAVLDNNSIERQH |
| | | AA* |
| 14 | GinA | MRPFANGELALHRRNNHQYGNRKIKHKRTRTLFYREGDIEYLAQQLFSQHYPFRK |
| | | WDDRPDSISGGPTQEEKDKFKDIARQQLSGWHPV* |

In some instances, a gene encoding a protein having an amino acid sequence with at least 70%, 75%, 80%, 85%, 90%, 95%, or 100% sequence identity to any one of the amino acid sequences in Table 1B is modified.

In some instances, the at least one gene encodes a protein with at least 70% h, 75%, 80%, 85%, 90%, 95%, or 100% sequence identity to SEQ ID NO: 9. In some instances, the at least one gene encodes a protein with at least 90% sequence identity to SEQ ID NO: 9. In some instances, the at least one gene encodes a protein with at least 70%, 75%, 80%, 85%, 90%, 95%, or 100% sequence identity to SEQ ID NO: 10. In some instances, the at least one gene encodes a protein with at least 90% sequence identity to SEQ ID NO: 10. In some instances, the at least one gene encodes a protein with at least 70%, 75%, 80%, 85%, 90%, 95%, or 100% sequence identity to SEQ ID NO: 11. In some instances, the at least one gene encodes a protein with at least 90% sequence identity to SEQ ID NO: 11. In some instances, the at least one gene encodes a protein with at least 70%, 75%, 80%, 85%, 90%, 95%, or 100% sequence identity to SEQ ID NO: 12. In some instances, the at least one gene encodes a protein with at least 90% sequence identity to SEQ ID NO: 12. In some instances, the at least one gene encodes a protein with at least 70%, 75%, 80%, 85%, 90%, 95%, or 100% sequence identity to SEQ ID NO: 13. In some instances, the at least one gene encodes a protein with at least 90% sequence identity to SEQ ID NO: 13. In some instances, the at least one gene encodes a protein with at least 70%, 75%, 80%, 85%, 90%, 95%, or 100% sequence identity to SEQ ID NO: 14. In some instances, the at least one gene encodes a protein with at least 90% sequence identity to SEQ ID NO: 14.

In some instances, the engineered bacterium comprises a reduced capacity for foaming behavior compared to a foaming behavior of a wild type AAB. In some instances, the engineered bacterium comprises a reduced capacity for foaming behavior at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to a foaming behavior of a reference bacterium (e.g., a wild type AAB or an AAB variant that does not comprise the modification described herein). In some instances, the engineered bacterium may comprise a reduced capacity for foaming behavior by at least about 5% to about 100% compared to a foaming behavior of a wild type AAB, or any range defined therein.

In some instances, the capacities for foaming behavior are measurable by detection of a foam layer produced by bacteria in a cell culture vessel (e.g., flask, bioreactor). A foam layer may be detected qualitatively by comparing images taken of foam layers produced by bacteria in different cell culture vessels.

### Cell Density

Volumetric productivity relates to the amount of product that can be produced per unit volume per unit time (e.g., g/L/h), whereas titer refers to the concentration of a product (e.g., g/L). Volumetric productivity provides insight into the efficiency of a given bioprocessing strategy. Increasing volumetric productivity may decrease the operational costs associated with a fermentation-based process.

Volumetric productivity of a fermentation may be increased by (i) improving the viable cell density of an engineered bacterium or (ii) by enhancing productivity of an engineered bacterium for a specific bioproduct. Cell-specific productivities for specific bioproducts can be manipulated case-by-case for an engineered bacterium through targeted metabolic engineering efforts. Comparatively, increasing the viable cell density of an engineered bacterium can improve volumetric productivity for many bioproducts produced by the engineered bacterium at the same time.

In some instances, the engineered bacterium comprises a modification of at least one gene to increase a capacity for cell density of the engineered bacterium. Non-limiting examples of genes that can be modified to increase cell density capacity include a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

In some instances, the engineered bacterium comprises an increased capacity for cell density compared to a capacity for cell density of a wild type AAB. In some instances, the engineered bacterium comprises a modification of at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein. In some instances, the modification comprises knockout, disruption, truncation, knockdown, or inhibition of the at least one gene.

In some instances, the engineered bacterium comprises a modification of a gene encoding N-Acyl-homoserine lactone acylase GqqA or a gene encoding N-Acyl-homoserine lactone lactonase Qsdr1. In some instances, the modification comprises enhancing expression of, heterologously expressing, or enhancing activity of a gene product (e.g., protein) associated with the at least one gene.

Modification of a gene that encodes N-Acyl-homoserine lactone-dependent transcriptional regulator (GinR (SEQ ID NO: 12)) (e.g., ΔginR) may decrease expression of GinA. Modification of a gene that encodes Acyl-homoserine-lactone synthase (GinI (SEQ ID NO: 13)) (e.g., Δginl) may decrease expression of GinA. Modification of a gene that encodes Acyl-homoserine-lactone response regulator (e.g., ΔginA) may improve cell density. Many Gram-negative bacteria use N-acylhomoserine lactone (AHL)-dependent quorum-sensing systems to regulate gene expression in concert with cell density. GinA (SEQ ID NO: 14) inhibits growth in media comprising ethanol. Thus, disruption of GinA (SEQ ID NO: 14) expression may increase cell density for bacteria grown in media comprising ethanol.

Modification of a gene that encodes Outer Membrane Protein A (OmpA)-like protein (e.g., ΔgmpA) may improve cell density. GinA (SEQ ID NO: 14) may activate expression of an OmpA-like protein (GmpA (SEQ ID NO: 15)), which itself inhibits cell growth. Modification of an GmpA-like homolog in the engineered bacterium may increase bacterial growth or increase cell density during fermentation.

Modification of N-Acyl-homoserine lactone acylase GqqA or N-Acyl-homoserine lactone lactonase Qsdr1 can depress activation of GinA (SEQ ID NO: 14)-dependent antifoaming repression. Many Gram-negative bacteria use N-acylhomoserine lactone (AHL)-dependent quorum-sensing systems to regulate gene expression. Expression of N-Acyl-homoserine lactone acylase GqqA or N-Acyl-homoserine lactone lactonase Qsdr1 may depress activation of GinA-dependent antifoaming repression via degradation of a N-Acyl-homoserine quorum-sensing signal molecule that activates GinA expression. In some instances, native N-Acyl-homoserine lactone acylase (GqqA (SEQ ID NO: 9)) expression may be enhanced. In some instances, recombinant N-Acyl-homoserine lactone lactonase Qsdr1 (SEQ ID NO: 10) from Sinorhizobium fredii NGR234 may be heterologously expressed. In some instances, the engineered bacterium is added as a microbial addition to a mixed culture (e.g., microbial community) of bacteria. Increased activity of N-Acyl-homoserine lactone acylase GqqA or N-Acyl-homoserine lactone lactonase Qsdr1 may increase the cell density of bacteria in the mixed culture.

TABLE 1C
Illustrative Proteins Relevant To Improving Cell Density

| SEQ ID | | |
|---|---|---|
| NO: | Identifier | Amino Acid Sequence |
| 9 | GqqA | MNGERIIAFQGRPGAYSDLACRQARPGWTTLPCQTFAQTIAAVHDGRAELAMLAC |
| | | ENSLAGRVPDIHALLPEAGLFIVGEHFQRVEHCLLGIPGSTLADARRIHTHPVAMA |
| | | QVRGIITELGLDPVVEFDTAGAAEMVREWGRKEDVAVASALAAELNGLEILRRNV |
| | | EDATHNTTRFYIASRRPATLPPPGPGFMTTLLFRVNNQPGALYKALGGLATAGVN |
| | | MTRLESYMLEGSFSATQFLMDVEGHPEAPPLARALDELSFFSEQQEILGVYPASPFR |
| | | RKP* |
| 10 | QsdR1 | MPHAETNRRTGQQETTMSLDNASRPSRSGRDELVPSRYAVKVGEIDVLVISDGVLP |
| | | IPTPVLATNADPAVRTAWLDDMFLPTDVLHWPLNVVLVRSGGQAILVDAGLGVEF |
| | | PDFPRAGQTVSRLEAAGVDLASVTDVVLTHMHMDHIGGLLADGVKDRLRPDLRI |
| | | HVAAAEVKFWEAPDFSHASMPSTVPPVLRRTAKRFMAEYQSQLRQFDEDYEVAP |
| | | GVVVTRTGGHTPGHSVVRLASGGDRLTFAGDAVFQVGFDHPDWHNGFEHDPEEA |
| | | ARVRVRLLRELAATREPLVATHLPFPSVCHVAADGDVFRWVPVVWDS* |
| 11 | WcaE | MAVSSNNAEMADDWATFDPAWYRARYAAMLDLMDIPVEQTREFHDAHGAALG |
| | | HAPNPFFDEEWYRATYPDVAAQVTAGTWRSGFDHYLNAGARTHNPHWLFDERA |
| | | YLAACPDITPATLDAGGYRNAYDHYLRVGDGEMRGGSCFFDPETCIALLAECDQD |
| | | GTRRPFAAYLRRGMDALPCRSVSLYFDAEWYVQAYPEAHAEITQGLWRSALHHY |
| | | LCNPTPQAFDPGPFFSESFYAMVNPDVQAAIEAGALRNGYAHFLSDGVHEQRKPC |
| | | STLDLAHYMRDPGVQADIAGRRARDGLGHYLAARPDLKPPPPPQVTEDQARALFR |
| | | HMCAARLPLLLDGRIDFTTDAPPALSVIIVAHDQFELTMSTLASLRANYHGPMQVV |
| | | VVDSGSRDGVAGIEDHVRGIEVLRFAGNIGFVRGCNAALRRVRAPATLYLNNDVD |
| | | LQYGAIANALSRLMADETTGAVGGRIIRTHGMLQEAGSMIWRDGSVQGYMRDAH |
| | | PCVPEAGFVRAVDFCSGVFLMVRTEVAQVLDGFDESYAPAYFEETDLCVRIRALG |
| | | YRILYDPAVCLVHYECGTSDGTSASRLIARNSDLFIRRHASGLRRRLLRHAPNEAR |
| | | ARHADDGRHILFIEDRLPLRHLGSGFTRSHDIVTTLAGLGYHVTIYPIFRPIEDAATR |
| | | AAAFPENVEVIHDRELPDLPAFLRARSGCFDAIWIARTHNAARVASILNDAASYIPA |
| | | GRIVVDTEALAACRDMEYDRLHAITPSRPLDARLADELRPLFVAQRVVAVNTAEA |
| | | DLLRGAGFDNVSVLGHVQAPRATGPGWAARRDILFLGAVHDMASPNLDSLAWFS |
| | | GQVLPLLVAQLGPDIRFSVCGHASPRVDLGPLRHHPNVRMLGRVADTAPVYDHH |
| | | RVFVAPTRYGAGIAYKLHEAAANGLPVVGSHLLCRQAGWRDGQDMLCASITDPA |
| | | DFARQVVRLYHDQVLWDTVRDNALTRITTEHAPQDYRNAVATIMDAVFTRG* |
| 12 | GinR | MVGWTIDLLAKLHDLENAATKHDLLSIYLDAVLSVGNVHVTIVELNRIEDPKENFI |
| | | HVGYPTEWVNFYIENNYIVSDPIIKKSRFMSHPYFWHEIRNINKSEKKIIRDVSEFGI |
| | | KKGLTIPVHTHDRLIYAICFAFTDKNVDQEIELYLRALSNFFIAGYKKLDEPTDMSP |
| | | PILTPREKECLRWTAKGKSSWETGMIVGVSERTVNFHINNALLKLKCTNRIMGVV |
| | | RAICAGLIEL* |
| 13 | GinI | MNDPLIQQEESGSMIEVVTVENAHWCGTALAEQFKFRYRHFVANEQWEVPFYKG |
| | | MEYDQPDTPAAVYLVWRDVAGVVRGMIRLLPTNRPYMLETLWPDMMPDPIVLP |
| | | GPTVWEITRFGVERNLSLSLRKQISAELILACIEFSVLNDIHTYLFLTAWGVLKRIVP |
| | | GAGVDAQIHSRKTLPSGHDVASAVVPVSQAVLDKARAKLNIHYAVLDNNSIERQH |
| | | AA* |
| 14 | GinA | MRPFANGELALHRRNNHQYGNRKIKHKRTRTLFYREGDIEYLAQQLFSQHYPFRK |
| | | WDDRPDSISGGPTQEEKDKFKDIARQQLSGWHPV* |
| 15 | GmpA | MRLRMVLLATALGAAPFATAMATTITGPYVDIGGGYDLTQTQHAHGFDKNQYEN |
| | | NANTAGYLDATDNARLLKEAHSRERMEHGDGWTGFATFGWGFGNGLRAEIEGD |
| | | YNWSALTGYNSVSGSAYGNNHQSGKSSGSDRSYGGFVNVLYDIDLKRLFNIDVPV |
| | | TPFVGVGAGYLWQNVDASTSVTRYLNVRQNGTNGSFAYQGIVGAAYDIPGVPGL |
| | | QMTTEYRMIGQVESFAMGNISQTGGGDRTLSYDHRFNHQFVGVRYAFNHAPPPPP |
| | | PAPAVAPPAPSAARTYLVFFDWDGAVLTDRARGIVAEAAQASTHVQTTRIEVNGY |
| | | TDNTSAHPGPRGEKYNLGLSMRRADSVKAELIRDGVPAGGIDIHGYGEAHPLVVT |
| | | QPDTREPQNRRVEIILH* |

In some instances, a gene encoding a protein having an amino acid sequence with at least 70%, 75%, 80%, 85%, 90%, 95%, or 100% sequence identity to any one of the amino acid sequences in Table 1C is modified.

In some instances, the at least one gene encodes a protein with at least 70%, 75%, 80%, 85%, 90%, 95%, or 100% sequence identity to SEQ ID NO: 9. In some instances, the at least one gene encodes a protein with at least 90% sequence identity to SEQ ID NO: 9. In some instances, the at least one gene encodes a protein with at least 70%, 75%, 80%, 85%, 90%, 95%, or 100% sequence identity to SEQ ID NO: 10. In some instances, the at least one gene encodes a protein with at least 90% sequence identity to SEQ ID NO: 10. In some instances, the at least one gene encodes a protein with at least 70%, 75%, 80%, 85%, 90%, 95%, or 100% sequence identity to SEQ ID NO: 11. In some instances, the at least one gene encodes a protein with at least 90% sequence identity to SEQ ID NO: 11. In some instances, the at least one gene encodes a protein with at least 70%, 75%, 80%, 85%, 90%, 95%, or 100% sequence identity to SEQ ID NO: 12. In some instances, the at least one gene encodes a protein with at least 90% sequence identity to SEQ ID NO: 12. In some instances, the at least one gene encodes a protein with at least 70%, 75%, 80%, 85%, 90%, 95%, or 100% sequence identity to SEQ ID NO: 13. In some instances, the at least one gene encodes a protein with at least 90% sequence identity to SEQ ID NO: 13. In some instances, the at least one gene encodes a protein with at least 70%, 75%, 80%, 85%, 90%, 95%, or 100% sequence identity to SEQ ID NO: 14. In some instances, the at least one gene encodes a protein with at least 90% sequence identity to SEQ ID NO: 14. In some instances, the at least one gene encodes a protein with at least 70%, 75%, 80%, 85%, 90%, 95%, or 100% sequence identity to SEQ ID NO: 15. In some instances, the at least one gene encodes a protein with at least 90% sequence identity to SEQ ID NO: 15.

In some instances, the engineered bacterium comprises an increased capacity for cell density compared to a capacity for cell density of said wild type AAB. In some instances, the engineered bacterium comprises an increased capacity for cell density by at least about 1%, 2% 3% 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to a capacity for cell density of a reference bacterium (e.g., a wild type AAB or an AAB variant that does not comprise the modification described herein). In some instances, the engineered bacterium may comprise an increased capacity for cell density by at least about 5% to about 100% compared to a capacity for cell density of said wild type AAB, or any range defined therein.

In some instances, the capacities for cell density are measured by detection of cell density of the bacteria in a cell culture vessel (e.g., flask, bioreactor). A capacity for cell density may be measured by detection of a wet weight for a sample of bacteria. A capacity for cell density may be measured by detection of the dry weight for a sample of bacteria. A capacity for cell density may be measured by counting the number of cells comprised in a sample of bacteria. A capacity for cell density is measured by detection of the optical density (OD) (e.g., absorbance at 600 nm across a 1 cm path length (OD₆₀₀)) corresponding to a sample of bacteria.

### Bioproducts

The engineered bacterium may produce a bioproduct. In some instances, the bioproduct may comprise acetic acid (e.g., vinegar), L-sorbose, gluconic acid, 2-keto-D-gluconate, 5-keto-D-gluconate, dihydroxyacetone (e.g., DHA), cellulose, or acetan. In some instances, the bioproduct comprises acetic acid. In some instances, the bioproduct may not comprise cellulose. The engineered bacterium may produce a plurality of bioproducts. In some instances, the plurality of bioproducts may comprise acetic acid (e.g., vinegar), L-sorbose, gluconic acid, 2-keto-D-gluconate, 5-keto-D-gluconate, dihydroxyacetone (e.g., DHA), cellulose, acetan, or a combination thereof. In some instances, the plurality of bioproducts may not comprise cellulose.

The engineered bacterium may enhance bioprocessing outcomes associated with the fermentative production of industrially relevant bioproducts. The features of the engineered bacterium (e.g., reduced cellulose formation, reduced foaming behavior, or increased cell density) may improve yield of the bioproduct by the engineered bacterium by improving bioavailability of cellular resources, avoiding downstream cellulose removal steps, avoiding physical and chemical foam mitigation treatment steps, or improving volumetric productivity of the engineered bacterium during fermentation.

The features of the engineered bacterium (e.g., reduced cellulose formation, reduced foaming behavior, or increased cell density) may improve yield of the bioproduct by the engineered bacterium by at least about 5%, 10%, 15%, 20%, 25%, 30%, 35%, 40%, 45%, 50%, 60%, 70%, 80%, 90%, 100%, 125%, 150%, 175%, 200%, 225%, 250%, 275%, 300%, 325%, 350%, 375%, 400%, 425%, 450%, 475%, 500%, or more, compared to yield by a reference bacterium (e.g., a wild type AAB or an AAB variant that does not comprise the modification or modifications described herein). Improved fermentation performance may be measured via yield (g/g product/substrate), product titer (g/L), productivity rate (g/L/h), or substrate (e.g., ethanol) consumption rate.

### Culture Conditions

The engineered bacterium may be grown in a range of temperature, humidity, light, or time conditions and with a variety of growth media. The engineered bacterium may be grown at a temperature of 30° C. for 72 hours, in acidic media (e.g., with a pH between 3 and 7).

In some instances, the engineered bacterium is grown at a temperature of at least about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., or greater. The engineered bacterium may be grown at a temperature of less than about 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., 15° C., or less. The engineered bacterium may be grown at a temperature of between about 15° C. to about 40° C., or any range defined therein. The engineered bacterium may be grown at a temperature of about 30° C.

In some instances, the engineered bacterium may be grown or fermented for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more, days. The engineered bacterium may be grown or fermented for less than about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 days. The engineered bacterium may be grown or fermented for between about 1 and 40 days, or any range defined therein. The engineered bacterium may be grown or fermented for 3 days (e.g., 72 hours).

In some instances, the engineered bacterium may be grown in media with a pH of at least about 3, 3.5, 4, 4.5, 5, 5.5, 6, 6.5, 7, or more. The engineered bacterium may be grown in media with a pH of at least about 7, 6.5, 6, 5.5, 5, 4.5, 4, 3.5, 3, or less. The engineered bacterium may be grown in media with a pH of between about 3 and 7, or any range defined therein.

In some instances, the engineered bacterium may be grown in media comprising a carbon source, a nitrogen source, minerals, amino acids, or vitamins, or any combination thereof. A carbon source may comprise a monosaccharide (e.g., glucose, mannose, fructose, arabinose, or xylose), a disaccharide (e.g., sucrose, maltose, or lactose), a trisaccharide (e.g., maltotriose), a polysaccharide (e.g., starch), a sugar alcohol (e.g., mannitol), an organic acid (e.g., citric acid, fumaric acid, acetic acid, lactic acid, or pyruvic acid), an alcohol (e.g., ethanol, or glycerol), or any combination thereof. A nitrogen source may comprise a yeast extract, a peptone, a tryptone, an amino acid, urea, an ammonium salt (e.g., ammonium sulfate, ammonia, or ammonium chloride), a nitrates, or any combination thereof. Minerals may comprise magnesium sulfate, magnesium chloride, potassium phosphate, potassium chloride, ferrous chloride, manganese chloride, manganese sulfate, zinc sulfate, zinc chloride, sodium molybdate, copper sulfate, calcium chloride, calcium carbonate, or any combination thereof. Vitamins may comprise biotin, thiamin, riboflavin, niacin, pyridoxine, folic acid, adenosyl-cobalamin or any combination thereof. In some instances, the media may comprise about 1 g/L dextrose monohydrate, about 0.5 g/L mineral salts (phosphates, potassium sulfate, magnesium sulfate, or a combination thereof), about 0.01 g/L vitamins, about 0.01 g/L trace minerals, about 0.015 g/L yeast extract, about 0.1-5% ethanol, and about 1-30% acetic acid. In some instances, the media may comprise 1 g/L dextrose monohydrate, 0.5 g/L mineral salts (phosphates, potassium sulfate, and magnesium sulfate), 0.01 g/L vitamins, 0.01 g/L trace minerals, 0.015 g/L yeast extract, 0.2-4% ethanol, and 2-24% acetic acid.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Certain Definitions

As used herein and in the appended claims, the singular forms “a,” “and,” and “the” include plural referents unless the context clearly dictates otherwise. Thus, for example, reference to “an agent” includes a plurality of such agents and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included.

The term “about” when referring to a number or a numerical range means that the number or numerical range referred to is an approximation within experimental variability (or within statistical experimental error), and thus the number or numerical range may vary between 1% and 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The term “modification” with respect to a gene or a nucleic acid (and related terms such as “modify” or “modified”) can mean knockout, disruption, truncation, knockdown, inhibition, insertion, deletion, mutation, or substitution. It can result in an increase or enhancement in expression of the modified gene. It can result in a decrease in expression of the modified gene.

### EXAMPLES

### Example 1: Method for Making an Engineered Bacterium Comprising a Modification

An engineered bacterium is made by modifying in the engineered bacterium: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; (d) at least one gene selected from the group consisting of: a gene encoding N-Acyl-homoserine lactone acylase GqqA and a gene encoding N-Acyl-homoserine lactone lactonase Qsdr1; (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein; or (f) a combination thereof. When the at least one gene is a natively expressed gene, the modifying comprises (a) knocking out, disrupting, knocking down, or inhibiting the natively expressed gene or (b) increasing expression of the natively expressed gene or increasing activity of a gene product (e.g., protein) associated with the natively expressed gene. When the at least one gene is a non-natively expressed gene, the modifying comprises heterologous expression of the non-natively expressed gene.

An unmodified bacterium is grown in media at 30° C. to mid-log phase. The media comprises about 10 g/L D-glucose, about 20 g/L Yeast Extract, about 20 g/L Peptone, about 6.76 g/L Na₂HPO₄×12H₂O, about 3.0 g/L Citric Acid, about 40 mL/L Ethanol, about 20 mL/L Acetic Acid. Mid-log phase of the unmodified bacteria is indicated by an optical density (OD₆₀₀) of about 0.6 to about 0.7. The unmodified bacteria is then washed cells several times with ice-cold 1 mM sterile HEPES buffer and resuspended in ice-cold 300 mM sterile sucrose.

The resuspended bacteria is then modified via a CRISPR/Cas system, a homologous recombination system, a phage recombinase system, a phage integrase system, or a transposase system. The modifying system is introduced to the unmodified bacteria by electroporation. More than 100 ng of the modifying system is added to 25 μL of the resuspended bacteria, then the modifying system and resuspended bacteria are transferred to a 0.1 cm electroporation cuvette. The cuvette is electroporated at 1,250 V, 25 uF, and 200 Ohms. The electroporated bacteria is recovered in room temperature media, then plated onto selective media plates. The plated bacteria is grown at 30° C. until colonies appear, then replated onto plates containing a selection agent or counter-selection agent. [0124] The engineered bacterium is a Komagataeibacter europaeus LMG 1521. The engineered bacterium is made by modifying a natively expressed sequence of interest via homologous recombination (e.g., via a vector comprising upstream and downstream homologous nucleotide sequences and genetic modification sequence for modifying sequence of interest). The modifying modifies sequence of interest, affecting activity of a gene product related to the sequence of interest in the engineered bacterium.

### Example 2: Method for Making an Engineered Bacterium Comprising a Modified bcsABCD

An engineered bacterium is made by modifying in the engineered bacterium a gene encoding at least one bacterial cellulose synthesis protein. The modifying comprises knocking out, disrupting, knocking down, or inhibiting the gene encoding at least one bacterial cellulose synthesis protein, and thus decreasing activity of a gene product (e.g., at least one bacterial cellulose synthesis protein) associated with the gene encoding at least one bacterial cellulose synthesis protein.

An unmodified bacterium is grown in media at 30° C. to mid-log phase. The media comprises about 10 g/L D-glucose, about 20 g/L Yeast Extract, about 20 g/L Peptone, about 6.76 g/L Na₂HPO₄×12H₂O, about 3.0 g/L Citric Acid, about 40 mL/L Ethanol, about 20 mL/L Acetic Acid. Mid-log phase of the unmodified bacteria is indicated by an optical density (OD₆₀₀) of about 0.6 to about 0.7. The unmodified bacteria is then washed cells several times with ice-cold 1 mM sterile HEPES buffer and resuspended in ice-cold 300 mM sterile sucrose.

The resuspended bacteria is then modified via a CRISPR/Cas system, a homologous recombination system, a phage recombinase system, a phage integrase system, or a transposase system. The modifying system is introduced to the unmodified bacteria by electroporation. More than 100 ng of the modifying system is added to 25 μL of the resuspended bacteria, then the modifying system and resuspended bacteria are transferred to a 0.1 cm electroporation cuvette. The cuvette is electroporated at 1,250 V, 25 uF, and 200 Ohms. The electroporated bacteria is recovered in room temperature media, then plated onto selective media plates. The plated bacteria is grown at 30° C. until colonies appear, then replated onto plates containing a selection agent or counter-selection agent.

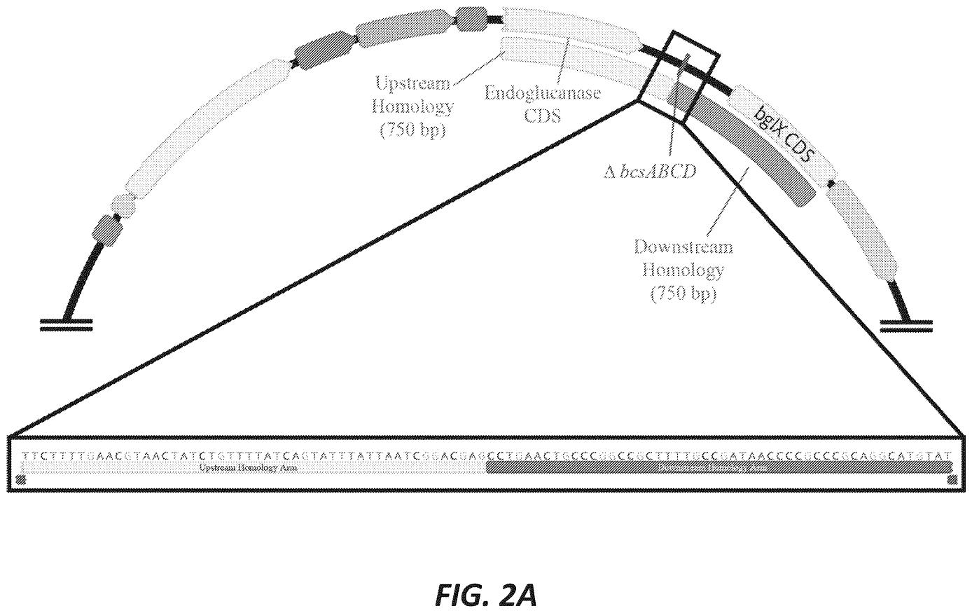

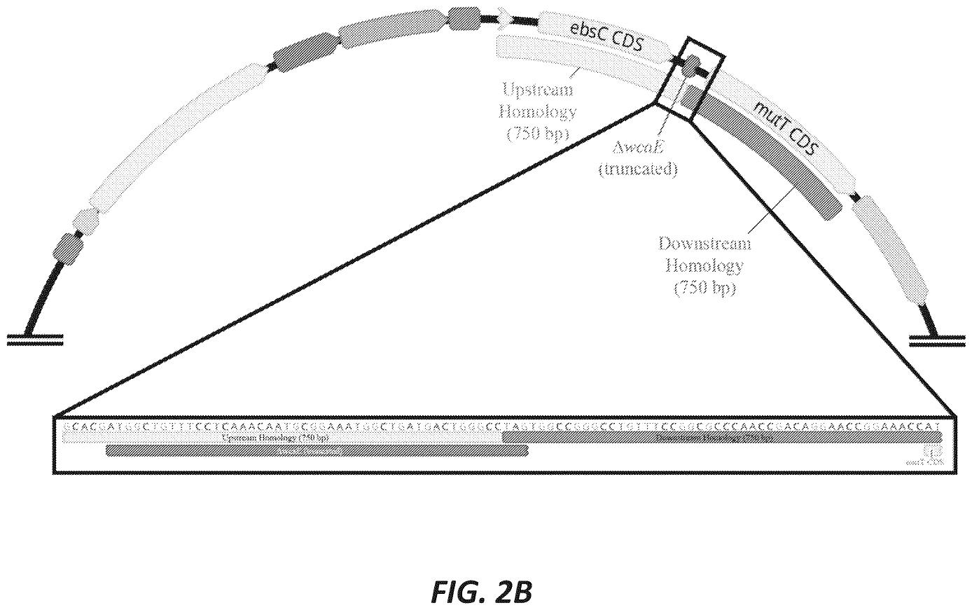

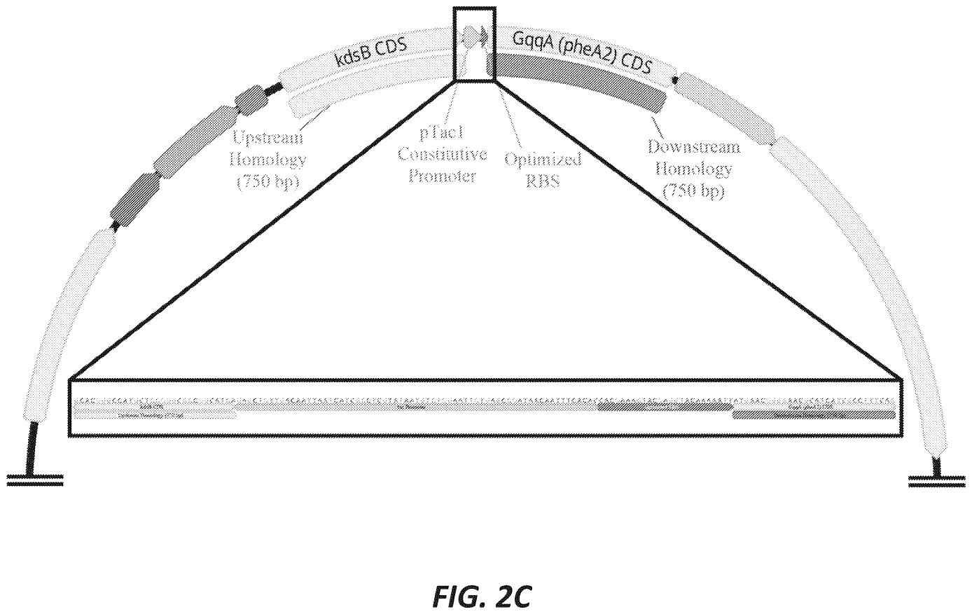

The engineered bacterium is a Komagataeibacter europaeus LMG 1521. The engineered bacterium is made by modifying a natively expressed bacterial cellulose synthase catalytic subunit A (SEQ ID NO: 1), bacterial cellulose synthase subunit B (SEQ ID NO: 2), bacterial cellulose synthase subunit C (SEQ ID NO: 3), and bacterial cellulose synthase subunit D (SEQ ID NO: 4), respectively, via homologous recombination (e.g., via a vector comprising a genetic modification sequence for deleting bcsABCD (FIG. 2A)). The modifying eliminates the natively expressed bcsABCD gene, rendering the bcsABCD gene product (e.g., bacterial cellulose synthase catalytic subunit A (SEQ ID NO: 1), bacterial cellulose synthase subunit B (SEQ ID NO: 2), bacterial cellulose synthase subunit C (SEQ ID NO: 3), and bacterial cellulose synthase subunit D (SEQ ID NO: 4)) non-active. Thus, the modification knocks out bacterial cellulose synthase subunit A, B, C, and D activity in the engineered bacterium.

### Example 3: Method for Making an Engineered Bacterium Comprising a Modified wcaE

An engineered bacterium is made by modifying in the engineered bacterium a gene encoding a GT2 family Glycosyltransferase (e.g., wcaE). The modifying comprises knocking out, disrupting, knocking down, or inhibiting the gene encoding a GT2 family Glycosyltransferase, and thus decreasing activity of a gene product (e.g., WcaE) associated with the gene encoding a GT2 family Glycosyltransferase.

An unmodified bacterium is grown in media at 30° C. to mid-log phase. The media comprises about 10 g/L D-glucose, about 20 g/L Yeast Extract, about 20 g/L Peptone, about 6.76 g/L Na₂HPO₄×12H₂O, about 3.0 g/L Citric Acid, about 40 mL/L Ethanol, about 20 mL/L Acetic Acid. Mid-log phase of the unmodified bacteria is indicated by an optical density (OD₆₀₀) of about 0.6 to about 0.7. The unmodified bacteria is then washed cells several times with ice-cold 1 mM sterile HEPES buffer and resuspended in ice-cold 300 mM sterile sucrose.

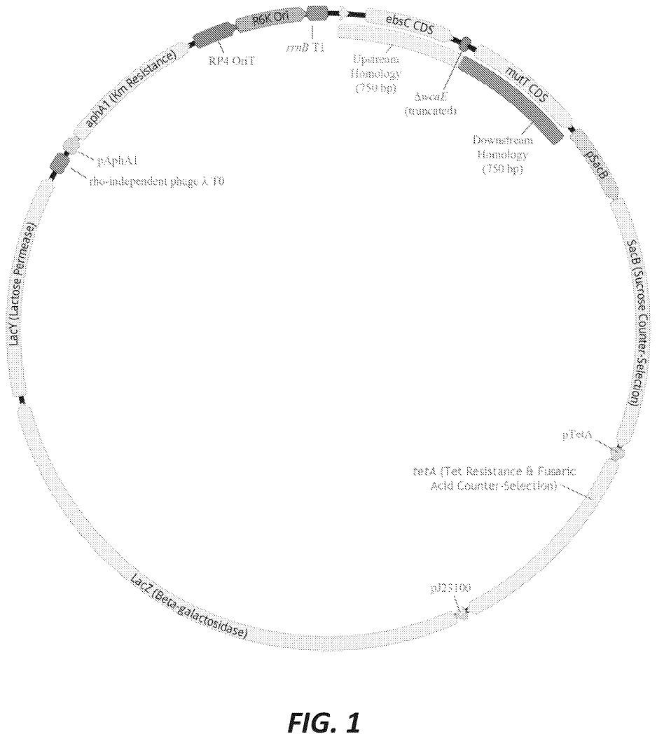

The resuspended bacteria is then modified via a homologous recombination system (FIG. 1 and FIG. 2B). The modifying system is introduced to the unmodified bacteria by electroporation. More than 100 ng of the modifying system is added to 25 μL of the resuspended bacteria, then the modifying system and resuspended bacteria are transferred to a 0.1 cm electroporation cuvette. The cuvette is electroporated at 1,250 V, 25 uF, and 200 Ohms. The electroporated bacteria is recovered in room temperature media, then plated onto selective media plates. The plated bacteria is grown at 30° C. until colonies appear, then replated onto plates containing a selection agent or counter-selection agent.

The engineered bacterium is a Komagataeibacter europaeus LMG 1521. The engineered bacterium is made by modifying a natively expressed wcaE (SEQ ID NO: 11) via homologous recombination (e.g., via a vector comprising a genetic modification sequence for truncating wcaE (FIG. 1 and FIG. 2B)). The modifying truncates the natively expressed wcaE gene, rendering the wcaE gene product (e.g., WcaE) non-active. Thus, the modification knocks out WcaE activity in the engineered bacterium.

### Example 4: Method for Making an Engineered Bacterium Comprising a Modified gqqA

An engineered bacterium is made by modifying in the engineered bacterium a gene encoding N-Acyl-homoserine lactone acylase GqqA (SEQ ID NO: 9) (e.g., gqqA). The modifying comprises increasing expression of the gene encoding N-Acyl-homoserine lactone acylase GqqA or increasing activity of a gene product (e.g., GqqA) associated with gqqA.

An unmodified bacterium is grown in media at 30° C. to mid-log phase. The media comprises about 10 g/L D-glucose, about 20 g/L Yeast Extract, about 20 g/L Peptone, about 6.76 g/L Na₂HPO₄×12H₂O, about 3.0 g/L Citric Acid, about 40 mL/L Ethanol, about 20 mL/L Acetic Acid. Mid-log phase of the unmodified bacteria is indicated by an optical density (OD₆₀₀) of about 0.6 to about 0.7. The unmodified bacteria is then washed cells several times with ice-cold 1 mM sterile HEPES buffer and resuspended in ice-cold 300 mM sterile sucrose.

The resuspended bacteria is then modified via a homologous recombination system (FIG. 1 and FIG. 2B). The modifying system is introduced to the unmodified bacteria by electroporation. More than 100 ng of the modifying system is added to 25 μL of the resuspended bacteria, then the modifying system and resuspended bacteria are transferred to a 0.1 cm electroporation cuvette. The cuvette is electroporated at 1,250 V, 25 uF, and 200 Ohms. The electroporated bacteria is recovered in room temperature media, then plated onto selective media plates. The plated bacteria is grown at 30° C. until colonies appear, then replated onto plates containing a selection agent or counter-selection agent.

The engineered bacterium is a Komagataeibacter europaeus LMG 1521. The engineered bacterium is made by modifying a natively expressed gqqA (SEQ ID NO: 9) via homologous recombination (e.g., via a vector comprising a genetic modification sequence for enhancing gqqA expression (FIG. 2C)). The modifying inserts a tac promoter and an optimized RBS upstream of the natively expressed gqqA gene, enhancing production of the gqqA gene product (e.g., GqqA). Thus, the modification increases GqqA activity in the engineered bacterium.

### Example 5: Method for Making an Engineered Bacterium Comprising a Modified bcsABCD, wcaE, and gqqA

An engineered bacterium is made by modifying in the engineered bacterium a gene encoding at least one bacterial cellulose synthesis protein, a gene encoding a GT2 family Glycosyltransferase (e.g., wcaE), and a gene encoding N-Acyl-homoserine lactone acylase GqqA (e.g., gqqA). Modifying the gene encoding at least one bacterial cellulose synthesis protein comprises knocking out, disrupting, knocking down, or inhibiting the gene encoding at least one bacterial cellulose synthesis protein, and thus decreasing activity of a gene product (e.g., at least one bacterial cellulose synthesis protein) associated with the gene encoding at least one bacterial cellulose synthesis protein. Modifying the gene encoding the GT2 family Glycosyltransferase comprises knocking out, disrupting, knocking down, or inhibiting the gene encoding a GT2 family Glycosyltransferase, and thus decreasing activity of a gene product (e.g., WcaE) associated with the gene encoding a GT2 family Glycosyltransferase. Modifying the gene encoding N-Acyl-homoserine lactone acylase GqqA comprises increasing expression of the gene encoding N-Acyl-homoserine lactone acylase GqqA or increasing activity of a gene product (e.g., GqqA) associated with the gene encoding N-Acyl-homoserine lactone acylase GqqA.

An unmodified bacterium is grown in media at 30° C. to mid-log phase. The media comprises about 10 g/L D-glucose, about 20 g/L Yeast Extract, about 20 g/L Peptone, about 6.76 g/L Na₂HPO₄×12H₂O, about 3.0 g/L Citric Acid, about 40 mL/L Ethanol, about 20 mL/L Acetic Acid. Mid-log phase of the unmodified bacteria is indicated by an optical density (OD₆₀₀) of about 0.6 to about 0.7. The unmodified bacteria is then washed cells several times with ice-cold 1 mM sterile HEPES buffer and resuspended in ice-cold 300 mM sterile sucrose.

The resuspended bacteria is then iteratively modified via a homologous recombination system (FIG. 1 and FIGS. 2A-C). The modifying system is introduced to the unmodified bacteria by electroporation. More than 100 ng of the modifying system is added to 25 μL of the resuspended bacteria, then the modifying system and resuspended bacteria are transferred to a 0.1 cm electroporation cuvette. The cuvette is electroporated at 1,250 V, 25 uF, and 200 Ohms. The electroporated bacteria is recovered in room temperature media, then plated onto selective media plates. The plated bacteria is grown at 30° C. until colonies appear, then replated onto plates containing a selection agent or counter-selection agent. This process is iterated with three vectors (FIGS. 2A-C) until the modified bacteria comprises modifications to a gene encoding at least one bacterial cellulose synthesis protein, a gene encoding a GT2 family Glycosyltransferase (e.g., wcaE), and a gene encoding N-Acyl-homoserine lactone acylase GqqA (e.g., gqqA).

The engineered bacterium is a Komagataeibacter europaeus LMG 1521. The engineered bacterium is made by modifying a natively expressed bacterial cellulose synthase catalytic subunit A (SEQ ID NO: 1), bacterial cellulose synthase subunit B (SEQ ID NO: 2), bacterial cellulose synthase subunit C (SEQ ID NO: 3), and bacterial cellulose synthase subunit D (SEQ ID NO: 4), respectively, via homologous recombination (e.g., via a vector comprising a genetic modification sequence for deleting bcsABCD (FIG. 2A)), by modifying a natively expressed wcaE (SEQ ID NO: 11) via homologous recombination (e.g., via a vector comprising a genetic modification sequence for truncating wcaE (FIG. 1 and FIG. 2B)), and by modifying a natively expressed gqqA (SEQ ID NO: 9) via homologous recombination (e.g., via a vector comprising a genetic modification sequence for enhancing gqqA expression (FIG. 2C)). The modifying eliminates the natively expressed bcsABCD gene, rendering the bcsABCD gene product (e.g., bacterial cellulose synthase catalytic subunit A (SEQ ID NO: 1), bacterial cellulose synthase subunit B (SEQ ID NO: 2), bacterial cellulose synthase subunit C (SEQ ID NO: 3), and bacterial cellulose synthase subunit D (SEQ ID NO: 4)) non-active. The modifying truncates the natively expressed wcaE gene, rendering the wcaE gene product (e.g., WcaE) non-active. The modifying inserts a tac promoter and an optimized RBS upstream of the natively expressed gqqA gene, enhancing production of the gqqA gene product (e.g., GqqA). Thus, the modification knocks out bacterial cellulose synthase subunit A, B, C, and D activity, knocks out WcaE activity, and increases GqqA activity in the engineered bacterium.

### Example 6: Method for Improved Bioproduct Production with an Engineered Bacterium Comprising a Modification

An engineered bacterium is used to produce a bioproduct. The engineered bacterium is a Komagataeibacter europaeus LMG 1521. The engineered bacterium comprises a modification of: (a) at least one gene selected from the group consisting of: a gene encoding a bacterial cellulose synthesis protein, a gene encoding phosphoglucomutase, a gene encoding a DTP-glucose-1-phosphate uridylyltransferase, and a gene encoding a diguanylate cyclase; (b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase Qsdr1; (c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator; (d) at least one gene selected from the group consisting of: a gene encoding N-Acyl-homoserine lactone acylase GqqA and a gene encoding N-Acyl-homoserine lactone lactonase Qsdr1; (e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein; or (f) a combination thereof. When the at least one gene is a natively expressed gene, the modifying comprises (a) knocking out, disrupting, knocking down, or inhibiting the natively expressed gene or (b) increasing expression of the natively expressed gene or increasing activity of a gene product (e.g., protein) associated with the natively expressed gene. When the at least one gene is a non-natively expressed gene, the modifying comprises heterologous expression of the non-natively expressed gene. The engineered bacterium produces acetic acid.

The engineered bacterium is grown (e.g., fermented) at 30° C. for least 72 hours in an acidic media (e.g., with a pH between 3 and 7) in a cell culture vessel (e.g., flask, bioreactor). The media comprises a carbon source, a nitrogen source, minerals, amino acids, or vitamins. The media comprises 1 g/L dextrose monohydrate, 0.5 g/L mineral salts (phosphates, potassium sulfate, and magnesium sulfate), 0.01 g/L vitamins, 0.01 g/L trace minerals, 0.015 g/L yeast extract, 0.2-4% ethanol, and 2-24% acetic acid.

In parallel, a reference bacterium (e.g., a wild type AAB or an AAB variant that does not comprise a modification of a natively encoded wcaE gene) is grown under the same conditions (e.g., at 30° C. for least 72 hours in an acidic media comprising a carbon source, a nitrogen source, minerals, amino acids, or vitamins) in a separate culture vessel.

Periodically during fermentation and after fermentation, the capacities of the engineered bacterium and the reference bacterium for cellulose formation are measured by detection of cellulose produced by each bacterium in the respective cell culture vessels. The cellulose produced by each bacterium is detected by sampling each fermentation broth, treating the samples with cellulase, and measuring glucose produced by the cellulase treatment via high-performance liquid chromatography (HPLC). Each glucose measurement indicates how much cellulose is in each sample. Samples with lower glucose measurements indicate the corresponding bacterium has a relatively reduced capacity for cellulose production. The absence of cellulose formation in the engineered bacterium is also assessed as the qualitative loss of cellulose aggregate accumulation in the cell culture vessel, as compared to aggregates observed in the culture containing the reference bacterium grown in the absence of cellulase.

Periodically during fermentation and after fermentation, the capacities of the engineered bacterium and the reference bacterium for foaming behavior is measured by detection of a foam layer produced by each bacterium in the respective cell culture vessels. A foam layer of each bacterium is detected qualitatively by taking images of each foam layer and comparing the images.

Periodically during fermentation and after fermentation, the capacities of the engineered bacterium and the reference bacterium for cell density are measured by detection of cell density of each bacterium in the respective cell culture vessels. The cell density of each bacterium is measured by detection of the dry weight for a sample of each bacterium or by detection of the optical density (OD) (e.g., absorbance at 600 nm across a 1 cm path length (OD₆₀₀)) of to a sample of each bacterium.

Following fermentation, the ethanol and acetic acid titers in the fermentation broth of the engineered bacterium and the reference bacterium are measured by high-performance liquid chromatography (HPLC).

The engineered bacterium demonstrates reduced cellulose formation, reduced foaming behavior, or increased cell density with respect to the reference bacterium. The engineered bacterium demonstrates a higher yield (g/g), higher titer (g/L), or increased productivity rate (g/L/h) of acetic acid compared to the reference bacterium.

### Example 7: Method for Improved Bioproduct Production with an Engineered Bacterium Comprising a Modified bcsABCD

An engineered bacterium is used to produce a bioproduct. The engineered bacterium is a Komagataeibacter europaeus LMG 1521. The engineered bacterium comprises a truncation of a natively expressed bcsABCD gene, rendering the bcsABCD gene product (e.g., bacterial cellulose synthase catalytic subunit A (SEQ ID NO: 1), bacterial cellulose synthase subunit B (SEQ ID NO: 2), bacterial cellulose synthase subunit C (SEQ ID NO: 3), and bacterial cellulose synthase subunit D (SEQ ID NO: 4)) non-active. The engineered bacterium produces acetic acid.

The engineered bacterium is grown (e.g., fermented) at 30° C. for least 72 hours in an acidic media (e.g., with a pH between 3 and 7) in a cell culture vessel (e.g., flask, bioreactor). The media comprises a carbon source, a nitrogen source, minerals, amino acids, or vitamins. The media comprises 1 g/L dextrose monohydrate, 0.5 g/L mineral salts (phosphates, potassium sulfate, and magnesium sulfate), 0.01 g/L vitamins, 0.01 g/L trace minerals, 0.015 g/L yeast extract, 0.2-4% ethanol, and 2-24% acetic acid.

In parallel, a reference bacterium (e.g., a wild type AAB or an AAB variant that does not comprise a modification of a natively encoded bcsABCD gene) is grown under the same conditions (e.g., at 30° C. for least 72 hours in an acidic media comprising a carbon source, a nitrogen source, minerals, amino acids, or vitamins) in a separate culture vessel.

Periodically during fermentation and after fermentation, the capacities of the engineered bacterium and the reference bacterium for cellulose formation are measured by detection of cellulose produced by each bacterium in the respective cell culture vessels. The cellulose produced by each bacterium is detected by sampling each fermentation broth, treating the samples with cellulase, and measuring glucose produced by the cellulase treatment via high-performance liquid chromatography (HPLC). Each glucose measurement indicates how much cellulose is in each sample. Samples with lower glucose measurements indicate the corresponding bacterium has a relatively reduced capacity for cellulose production. The absence of cellulose formation in the engineered bacterium is also assessed as the qualitative loss of cellulose aggregate accumulation in the cell culture vessel, as compared to aggregates observed in the culture containing the reference bacterium grown in the absence of cellulase.

Periodically during fermentation and after fermentation, the capacities of the engineered bacterium and the reference bacterium for foaming behavior is measured by detection of a foam layer produced by each bacterium in the respective cell culture vessels. A foam layer of each bacterium is detected qualitatively by taking images of each foam layer and comparing the images.

Periodically during fermentation and after fermentation, the capacities of the engineered bacterium and the reference bacterium for cell density are measured by detection of cell density of each bacterium in the respective cell culture vessels. The cell density of each bacterium is measured by detection of the dry weight for a sample of each bacterium or by detection of the optical density (OD) (e.g., absorbance at 600 nm across a 1 cm path length (OD₆₀₀)) of to a sample of each bacterium.

Following fermentation, the ethanol and acetic acid titers in the fermentation broth of the engineered bacterium and the reference bacterium are measured by high-performance liquid chromatography (HPLC).

The engineered bacterium demonstrates reduced cellulose formation with respect to the reference bacterium. The engineered bacterium demonstrates a higher yield (g/g), higher titer (g/L), or increased productivity rate (g/L/h) of acetic acid compared to the reference bacterium.

### Example 8: Method for Improved Bioproduct Production with an Engineered Bacterium Comprising a Modified wcaE

An engineered bacterium is used to produce a bioproduct. The engineered bacterium is a Komagataeibacter europaeus LMG 1521. The engineered bacterium comprises a truncation of a natively expressed wcaE gene, rendering the wcaE gene product (e.g., WcaE (SEQ ID NO: 11)) non-active. The engineered bacterium produces acetic acid.

The engineered bacterium is grown (e.g., fermented) at 30° C. for least 72 hours in an acidic media (e.g., with a pH between 3 and 7) in a cell culture vessel (e.g., flask, bioreactor). The media comprises a carbon source, a nitrogen source, minerals, amino acids, or vitamins. The media comprises 1 g/L dextrose monohydrate, 0.5 g/L mineral salts (phosphates, potassium sulfate, and magnesium sulfate), 0.01 g/L vitamins, 0.01 g/L trace minerals, 0.015 g/L yeast extract, 0.2-4% ethanol, and 2-24% acetic acid.

In parallel, a reference bacterium (e.g., a wild type AAB or an AAB variant that does not comprise a modification of a natively encoded wcaE gene) is grown under the same conditions (e.g., at 30° C. for least 72 hours in an acidic media comprising a carbon source, a nitrogen source, minerals, amino acids, or vitamins) in a separate culture vessel.

Periodically during fermentation and after fermentation, the capacities of the engineered bacterium and the reference bacterium for cellulose formation are measured by detection of cellulose produced by each bacterium in the respective cell culture vessels. The cellulose produced by each bacterium is detected by sampling each fermentation broth, treating the samples with cellulase, and measuring glucose produced by the cellulase treatment via high-performance liquid chromatography (HPLC). Each glucose measurement indicates how much cellulose is in each sample. Samples with lower glucose measurements indicate the corresponding bacterium has a relatively reduced capacity for cellulose production. The absence of cellulose formation in the engineered bacterium is also assessed as the qualitative loss of cellulose aggregate accumulation in the cell culture vessel, as compared to aggregates observed in the culture containing the reference bacterium grown in the absence of cellulase.

Periodically during fermentation and after fermentation, the capacities of the engineered bacterium and the reference bacterium for foaming behavior is measured by detection of a foam layer produced by each bacterium in the respective cell culture vessels. A foam layer of each bacterium is detected qualitatively by taking images of each foam layer and comparing the images.

Periodically during fermentation and after fermentation, the capacities of the engineered bacterium and the reference bacterium for cell density are measured by detection of cell density of each bacterium in the respective cell culture vessels. The cell density of each bacterium is measured by detection of the dry weight for a sample of each bacterium or by detection of the optical density (OD) (e.g., absorbance at 600 nm across a 1 cm path length (OD₆₀₀)) of to a sample of each bacterium.

Following fermentation, the ethanol and acetic acid titers in the fermentation broth of the engineered bacterium and the reference bacterium are measured by high-performance liquid chromatography (HPLC).

The engineered bacterium demonstrates reduced foaming behavior or increased cell density with respect to the reference bacterium. The engineered bacterium demonstrates a higher yield (g/g), higher titer (g/L), or increased productivity rate (g/L/h) of acetic acid compared to the reference bacterium.

### Example 9: Method for Improved Bioproduct Production with an Engineered Bacterium Comprising a Modified gqqA

An engineered bacterium is used to produce a bioproduct. The engineered bacterium is a Komagataeibacter europaeus LMG 1521. The engineered bacterium comprises a insertion of a promoter and RBS upstream of a natively expressed gqqA gene, rendering the gqqA gene product (e.g., N-Acyl-homoserine lactone acylase GqqA (SEQ ID NO: 9)) more active. The engineered bacterium produces acetic acid.

The engineered bacterium is grown (e.g., fermented) at 30° C. for least 72 hours in an acidic media (e.g., with a pH between 3 and 7) in a cell culture vessel (e.g., flask, bioreactor). The media comprises a carbon source, a nitrogen source, minerals, amino acids, or vitamins. The media comprises 1 g/L dextrose monohydrate, 0.5 g/L mineral salts (phosphates, potassium sulfate, and magnesium sulfate), 0.01 g/L vitamins, 0.01 g/L trace minerals, 0.015 g/L yeast extract, 0.2-4% ethanol, and 2-24% acetic acid.

In parallel, a reference bacterium (e.g., a wild type AAB or an AAB variant that does not comprise a modification of a natively encoded gqqA gene) is grown under the same conditions (e.g., at 30° C. for least 72 hours in an acidic media comprising a carbon source, a nitrogen source, minerals, amino acids, or vitamins) in a separate culture vessel.

Periodically during fermentation and after fermentation, the capacities of the engineered bacterium and the reference bacterium for cellulose formation are measured by detection of cellulose produced by each bacterium in the respective cell culture vessels. The cellulose produced by each bacterium is detected by sampling each fermentation broth, treating the samples with cellulase, and measuring glucose produced by the cellulase treatment via high-performance liquid chromatography (HPLC). Each glucose measurement indicates how much cellulose is in each sample. Samples with lower glucose measurements indicate the corresponding bacterium has a relatively reduced capacity for cellulose production. The absence of cellulose formation in the engineered bacterium is also assessed as the qualitative loss of cellulose aggregate accumulation in the cell culture vessel, as compared to aggregates observed in the culture containing the reference bacterium grown in the absence of cellulase.

Periodically during fermentation and after fermentation, the capacities of the engineered bacterium and the reference bacterium for foaming behavior is measured by detection of a foam layer produced by each bacterium in the respective cell culture vessels. A foam layer of each bacterium is detected qualitatively by taking images of each foam layer and comparing the images.

Periodically during fermentation and after fermentation, the capacities of the engineered bacterium and the reference bacterium for cell density are measured by detection of cell density of each bacterium in the respective cell culture vessels. The cell density of each bacterium is measured by detection of the dry weight for a sample of each bacterium or by detection of the optical density (OD) (e.g., absorbance at 600 nm across a 1 cm path length (OD₆₀₀)) of to a sample of each bacterium.

Following fermentation, the ethanol and acetic acid titers in the fermentation broth of the engineered bacterium and the reference bacterium are measured by high-performance liquid chromatography (HPLC).

The engineered bacterium demonstrates reduced cellulose formation, reduced foaming behavior, and increased cell density with respect to the reference bacterium. The engineered bacterium demonstrates a higher yield (g/g), higher titer (g/L), or increased productivity rate (g/L/h) of acetic acid compared to the reference bacterium.

### Example 10: Method for Improved Bioproduct Production with an Engineered Bacterium Comprising a Modified bcsABCD, wcaE, and gqqA

An engineered bacterium is used to produce a bioproduct. The engineered bacterium is a Komagataeibacter europaeus LMG 1521. The engineered bacterium comprises a truncation of a natively expressed bcsABCD gene, rendering the bcsABCD gene product (e.g., bacterial cellulose synthase catalytic subunit A (SEQ ID NO: 1), bacterial cellulose synthase subunit B (SEQ ID NO: 2), bacterial cellulose synthase subunit C (SEQ ID NO: 3), and bacterial cellulose synthase subunit D (SEQ ID NO: 4)) non-active; a truncation of a natively expressed wcaE gene, rendering the wcaE gene product (e.g., WcaE (SEQ ID NO: 11)) non-active; and insertion of a promoter and RBS upstream of a natively expressed gqqA gene, rendering the gqqA gene product (e.g., N-Acyl-homoserine lactone acylase GqqA (SEQ ID NO: 9)) more active. The engineered bacterium produces acetic acid.

The engineered bacterium is grown (e.g., fermented) at 30° C. for least 72 hours in an acidic media (e.g., with a pH between 3 and 7) in a cell culture vessel (e.g., flask, bioreactor). The media comprises a carbon source, a nitrogen source, minerals, amino acids, or vitamins. The media comprises 1 g/L dextrose monohydrate, 0.5 g/L mineral salts (phosphates, potassium sulfate, and magnesium sulfate), 0.01 g/L vitamins, 0.01 g/L trace minerals, 0.015 g/L yeast extract, 0.2-4% ethanol, and 2-24% acetic acid.

In parallel, a reference bacterium (e.g., a wild type AAB or an AAB variant that does not comprise a modification of a natively encoded gqqA gene) is grown under the same conditions (e.g., at 30° C. for least 72 hours in an acidic media comprising a carbon source, a nitrogen source, minerals, amino acids, or vitamins) in a separate culture vessel.

Periodically during fermentation and after fermentation, the capacities of the engineered bacterium and the reference bacterium for cellulose formation are measured by detection of cellulose produced by each bacterium in the respective cell culture vessels. The cellulose produced by each bacterium is detected by sampling each fermentation broth, treating the samples with cellulase, and measuring glucose produced by the cellulase treatment via high-performance liquid chromatography (HPLC). Each glucose measurement indicates how much cellulose is in each sample. Samples with lower glucose measurements indicate the corresponding bacterium has a relatively reduced capacity for cellulose production. The absence of cellulose formation in the engineered bacterium is also assessed as the qualitative loss of cellulose aggregate accumulation in the cell culture vessel, as compared to aggregates observed in the culture containing the reference bacterium grown in the absence of cellulase.

Periodically during fermentation and after fermentation, the capacities of the engineered bacterium and the reference bacterium for foaming behavior is measured by detection of a foam layer produced by each bacterium in the respective cell culture vessels. A foam layer of each bacterium is detected qualitatively by taking images of each foam layer and comparing the images.

Periodically during fermentation and after fermentation, the capacities of the engineered bacterium and the reference bacterium for cell density are measured by detection of cell density of each bacterium in the respective cell culture vessels. The cell density of each bacterium is measured by detection of the dry weight for a sample of each bacterium or by detection of the optical density (OD) (e.g., absorbance at 600 nm across a I cm path length (OD₆₀₀)) of to a sample of each bacterium.

Following fermentation, the ethanol and acetic acid titers in the fermentation broth of the engineered bacterium and the reference bacterium are measured by high-performance liquid chromatography (HPLC).

The engineered bacterium demonstrates reduced cellulose formation, reduced foaming behavior, and increased cell density with respect to the reference bacterium. The engineered bacterium demonstrates a higher yield (g/g), higher titer (g/L), or increased productivity rate (g/L/h) of acetic acid compared to the reference bacterium.

1. An engineered bacterium, comprising:
(a) a reduced capacity for cellulose formation compared to a capacity for cellulose formation of a wild type acetic acid bacterium (AAB),
(b) a reduced capacity for foaming behavior compared to a foaming behavior of said wild type AAB,
(c) an increased capacity for cell density compared to a capacity for cell density of said wild type AAB, or
(d) a combination thereof.

2. The engineered bacterium of claim 1, comprising a reduced capacity for cellulose formation compared to a capacity for cellulose formation of said wild type AAB.

3. The engineered bacterium of claim 2, comprising a modification of at least one gene selected from the group consisting of: a gene encoding a bacterial cellulose synthesis protein, a gene encoding phosphoglucomutase, a gene encoding a UTP-glucose-1-phosphate uridylyltransferase, and a gene encoding a diguanylate cyclase.

4. (canceled)

5. The engineered bacterium of claim 2, comprising a modification of at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase QsdR1.

6. (canceled)

7. The engineered bacterium of claim 1, comprising a reduced capacity for foaming behavior compared to a foaming behavior of said wild type AAB.

8. (canceled)

9. The engineered bacterium of claim 7, comprising a modification of at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator.

10. (canceled)

11. The engineered bacterium of claim 7, comprising a modification of a gene encoding N-Acyl-homoserine lactone acylase GqqA or a gene encoding N-Acyl-homoserine lactone lactonase QsdR1.

12. (canceled)

13. The engineered bacterium of claim 1, comprising an increased capacity for cell density compared to a capacity for cell density of a wild type AAB.

14. The engineered bacterium of claim 13, comprising a modification of at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

15. (canceled)

16. The engineered bacterium of claim 13, comprising a modification of a gene encoding N-Acyl-homoserine lactone acylase GqqA or a gene encoding N-Acyl-homoserine lactone lactonase QsdR1.

17. (canceled)

18. An engineered bacterium, comprising a modification of:
(a) at least one gene selected from the group consisting of: a gene encoding a bacterial cellulose synthesis protein, a gene encoding phosphoglucomutase, a gene encoding a DTP-glucose-I-phosphate uridylyltransferase, and a gene encoding a diguanylate cyclase;
(b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase QsdR1;
(c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator;
(d) at least one gene selected from the group consisting of: a gene encoding N-Acyl-homoserine lactone acylase GqqA and a gene encoding N-Acyl-homoserine lactone lactonase QsdR1;
(e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein; or
(f) a combination thereof.

19-43. (canceled)

44. The engineered bacterium of claim 18, comprising a modification of:
(a) at least one gene selected from the group consisting of: a gene encoding a bacterial cellulose synthesis protein, a gene encoding phosphoglucomutase, a gene encoding a DTP-glucose-I-phosphate uridylyltransferase, and a gene encoding a diguanylate cyclase;
(b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase QsdR1;
(c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator;
(d) at least one gene selected from the group consisting of: a gene encoding N-Acyl-homoserine lactone acylase GqqA and a gene encoding N-Acyl-homoserine lactone lactonase QsdR1; and
(e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein.

45-49. (canceled)

50. The engineered bacterium of claim 1, comprising said reduced capacity for cellulose formation by at least about 90% compared to said capacity for cellulose formation of said wild type AAB.

51-56. (canceled)

57. The engineered bacterium of claim 1, comprising said reduced capacity for foaming behavior by at least about 9% compared to a foaming behavior of said wild type AAB.

58-62. (canceled)

63. The engineered bacterium of claim 1, comprising said increased capacity for cell density by at least about 9% compared to a capacity for cell density of said wild type AAB.

64-69. (canceled)

70. The engineered bacterium of claim 1, wherein said engineered bacterium is of a genus Acetobacter, a genus Gluconacetobacter, a genus Gluconobacter, or a genus Komagataeibacter.

71. The engineered bacterium of claim 1, wherein said engineered bacterium is Komagataeibacter europaeus LMG 1521.

72-76. (canceled)

77. A method of making a bioproduct, comprising growing said engineered bacterium of claim 1.

78. The method of claim 77, wherein said bioproduct is acetic acid, L-sorbose, gluconic acid, 2-keto-D-gluconate, 5-keto-D-gluconate, dihydroxyacetone (DHA), cellulose, or acetan.

79. (canceled)

80. (canceled)

81. (canceled)

82. A method of making an engineered bacterium, the method comprising modifying in a bacterium:
(a) at least one gene selected from the group consisting of: a gene encoding a bacterial cellulose synthesis protein, a gene encoding phosphoglucomutase, a gene encoding a DTP-glucose-I-phosphate uridylyltransferase, and a gene encoding a diguanylate cyclase;
(b) at least one gene selected from the group consisting of: a gene encoding a soluble cellulase protein, a gene encoding endoglucanase CelY, a gene encoding endoglucanase CelZ, a gene encoding N-Acyl-homoserine lactone acylase GqqA, and a gene encoding N-Acyl-homoserine lactone lactonase QsdR1;
(c) at least one gene selected from the group consisting of: a gene encoding a GT2 family Glycosyltransferase, a gene encoding a N-Acyl-homoserine lactone-dependent transcriptional regulator, a gene encoding an Acyl-homoserine-lactone synthase, and a gene encoding an Acyl-homoserine-lactone response regulator;
(d) at least one gene selected from the group consisting of: a gene encoding N-Acyl-homoserine lactone acylase GqqA and a gene encoding N-Acyl-homoserine lactone lactonase QsdR1;
(e) at least one gene selected from the group consisting of: a gene encoding a N-Acyl-homoserine lactone-dependent transcriptional regulator, a gene encoding an Acyl-homoserine-lactone synthase, a gene encoding an Acyl-homoserine-lactone response regulator, and a gene encoding an Outer Membrane Protein A (OmpA)-like protein; or
(f) a combination thereof.

83-89. (canceled)

90. The method of claim 82, wherein said bacterium is of a genus Acetobacter, a genus Gluconacetobacter, a genus Gluconobacter, or a genus Komagataeibacter.

91. The method of claim 82, wherein said bacterium is Komagataeibacter europaeus LMG 1521.


SEQUENCE LISTING (15 sequences)

SEQ ID NO: 1 - protein, 745 aa, synthetic construct
MSEVQSSAPAESWFGRFSNKILSLRGASYIVGAVGLFALLAATIVTLSIHEQLIVALICVAVFFIVGRRKSRRTQVFLEVLSALVSLRYLTWRLTETLDFDTWIQGLLGVTLLLAELYALYMLFLSYFQTISPLHRAPLPLSPNPDEWPTVDIFIPTYDEDLSIVRLTVLAALGIDWPPEKMNVYILDDGKRETFGRFAEECGARYITRPDNTHYKAGNLNHAIKQTRGDFILILDCDHIPTRAFLQIAMGWMVEDPKIALMQTPHHFYSPDPFQRNLAVGYRTPPEGNLFYGVIQDGNDFWDATFFCGSCAILRRKAIEEIGGFATETVTEDAHTALRMQRKGWSTAYLRIPLASGLATERLITHIGQRMRWARGMIQIFRVDNPMLGPGLKLGQRLCYLSAMTSFFFAIPRVIFLASPLAFLFFNQNIIAASPLAVLAYAIPHMFHSIATAAKVNRGWRYSFWSEVYETVMALFLVRVTIVTMIFPSKGKFNVTEKGGVLENETFDLDATYPNIIFAVVMALGLLRGAYALIFQHLDIIAERAYALNCIWSVISLIILLAAIAVGRETKQIRHSHRIEAHIPVTVYDYEGNSSHGITEDVSMGGVAIHMPWRDVTPDQPVQVVVHAILEGEEVNLPATMIRSANGKAVFTWSINNIQIEASVVRFVFGRADAWLQWNNYEDDRPLRSLWSLILSIKALFRKKGQMIAHSRPKKKPIALPVERREPTTSQGGQKQEGKISRAAS

SEQ ID NO: 2 - protein, 807 aa, synthetic construct
MRPRDMKMVSLIALLVFATGAQAAPIASKAPVPQPTGNDLPPLPAAAPAAAQPQAQAGNNAAPAAAPAGDAGSNADAILDNAENATGASSDVATVHTYSLQELGAAGALTMRGTAPLQGLQFGIPADQLVTSARLVVSGAMSPNLQPDNSTVTITLNEQYIGTLRPDPSHPAFGPLSFDVNPIFFVSGNRLNFSFATGSKGCTDPSNGLQWASVSEHSQLQITTIPLPPRRQLARLPQPFFDKTVRQKVVIPFVLAQTFDPEVLKASGIIASWFGQQTDYRGVNFPVFSTIPQTGNAIVVGVADELPSALGRPSVSGPTLMEVANPSDPNGTLLLVTGRDRDEVITASKGIGFGSSALPVASRMDVAPIDVAPRVANDAPSFLPTTRPVRLGELVPVSALQGEGYTPGVLSVPFRVSPDLYTWRDRPYKLNVRFRAPDGPILDVARSHLDVGINNTYLQSYSLREQNSVVDQLLRRVGVGTQNAGVEQHTLTIPPWMVFGQDQLQFYFDAAPLTEPGCRPGPSLIHMSVDPDSTIDLSNAYHITRMPNLAYMASAGYPFTTYADLSRSAVVLPDHPNGTVVSAYLDLMGFMGATTWYPVSGVDIVSADHVSDVADRNLIVLSTLSNSGEVSSLLSNSAYQISDGRLHMGLRSTLSGVWNIFQDPMSVISNTHPTEVETTLTGGVGAMVEAESPLASGRTVLALLSGDGQGLDNLVQILGQRKNQAKVQGDLVLAHGDDLTSYRSSPLYTVGTLPMWLMPDWYMHNHPIRVVVVGLFGCLLVVAVLVRALFRHAMYRRRQLQEERQKS

SEQ ID NO: 3 - protein, 1331 aa, synthetic construct
MIMSRRYVFSLSAGLLASSCMGVMMAVPVARAQQASTAMTGAQATGGTAAPRQILLQQARFWLQQQQYDNARQALQNAQRIAPDSPDVLEVQGEYQTAIGNREAAADTLRHLQQVAPGSTAANSLTDLLHERSISTGDLSQVRALAASGHNEQAVAGYQKLFNGGKPPHSLAVEYYQTMAGVPADWDQARAGLASVVAANPQDYRAQLAFAQALTYNTPTRMEGLARLKDLEGFRAQAPVEAAAAAQSYRQTLSWLPVTPETQPLMQQWLAAHPNDTALKEHMLHPPGGPPDKAGLARQAGYQQLNAGRLSAAEQSFQSALQINAHDADSLGGMGLVSMRQGDAAEARRYFQEAMAADPKTADRWRPALAGMEISGDYAAVRQMIAAHQYTEAKQRLTSLARQPGQFTGATLMLADLQRTTGQIDASEQSYRSVLARDPNSQLALMGLARVDMAQGNTAEARQLLSRVGPQYAEQVGEIEVTGLMAAASHTSDSARKVSILREAMTQAPRDPWVRINLANALQQQGDVAEAGRVMQPILANPVTAQDRQAGILYTYGAGNDAATRRLLSGLSPEDYSPAIRSIATEMEIKEDLASRLSMVANPVPLIREALSPPDPTGARGVAVADLFRQRGDMLHARMALRIASTRTIDLSPDQRLAYATEYMKISNPVAAARLLAPLGDGSGSGMGNALPPEQQQTLQQLRMGISVAQSDLLNQRGDQAQAYDQLAPALRADPEATAPKLALARLYNGEGKASKALEIDLAVLRHNPQDLDARQAAVQAAVNSGHKSLATRLAMDGVQESPMDARAWLSMAVADQADGHGHRTIADLRRAYDLRLQQVEGSRMAAGAGAAGAEQDALAPPSSNPFRHHGYGRQTELGAPVTGGSYSMEATSPEASDQMLSSISGQINTLRENLAPSIDGGLGFRSRSGEHGMGRLTEANIPIVGRLPLQAGESSLTFSITPTMIWSGDLDSGSVYDVPRFGTDMATQAWNQYVNYMNRNNQGSTSRYKSQLVKGGEGEAGFAPDVQFGNSWVRADVGASPIGFPITNVLGGVEFSPRVGPVTFRVSAERRSITNSVLSYGGMRDPNYYSAIGRYARSLYGKELASQWSQEWGGVVTNHFHGQVEATLGNTIVYGGGGYAIQTGKNVRRNSEREAGIGVNTLVWHNANMLVRIGVSLTYFGYGNNQDFYTYGQGGYFSPQSYYAATVPIRYAGQHKRLDWDVNASVGYQVFHEHSSPFFPTSSLLQSGAQYVANNYVTNATSSDYLSQETVNSAYYPGDSVASLTGGFNARVGYRFTHNLRLDLSGRWQKAGNWTESGAMISAHYLIMDQ

SEQ ID NO: 4 - protein, 156 aa, synthetic construct
MTTFNAKPDFSLFLQALSWEIDDQAGIEVRNDLLREVGRGMAGRLQPPLCNTIHQLQIELNALLGMINWGYVKLELLAEEQAMRIVHEDLPQVGSAGEPSGTWLAPVLEGLYGRWITSOPGAFGDYVVSRDVDAEDLNSVPTOTVILYMRTRSNSN

SEQ ID NO: 5 - protein, 469 aa, synthetic construct
MIFRHRFDPTSLREYDIRGTVGKTLGPEDAYAIGRTFASVVAGDGGRTVVVGYDGRLSSPALERALVEGAMASGMDVVRIGCGPTPMLYFASADLNADGAVMVTGSHNPPDQNGFKIVLANRPFFGAQIAMLGQMAATGNVVAQASGTTRMVDIRAAYIDRLLRDHDHDARALNVVWDCGNGAAGDVLSLLVKRLPGRHRVLNAAIDGRFPAHHPDPTIPANLQQLITAVRQDGADLGIAFDGDADRLGVVDDTGAIVWADQLLLILARDMLRARPGATIIADIKTSQVVFDEIDRAGGRALMWKSGHSQMKERMAETGALLAGEMSGHLFFADRWYGFDDALYAALRLLDVISRLDGSLSDARRALPATVSTPELRFACPDTRKFDVITEVATRLAQAGADVCDIDGVRVSTPDGWWLLRASNTQAVLVARAEGATQAALDRLKAALSAQLAQSGVTLPDHADPATPH

SEQ ID NO: 6 - protein, 289 aa, synthetic construct
MIKPLKKAVLPVAGLGTRFLPATKCVPKEMLTVVDRPLIQYAIDEAREAGIEEFCLVSSRGKDSLIDYFDISYELEDTLKARKKTSALKALEATRVIPGTMLSVRQQEPLGLGHAIWCAREFIGNDPFAILLPDDVVQSKKSCIGQLVEVYNKTGGNVLAVTEVPREQTGSYGILDVGKDDGKTVEVKGLVEKPDPKDAPSTLSVIGRYVLTADVLKHLAKLEKGAGGEVQLTDAMAKTIGHVPFHGYRYEGKRFDCGSKIGFLEAQIAFALEREELAPGVREFLTKYK

SEQ ID NO: 7 - protein, 332 aa, synthetic construct
MGKPMWRCWALMLMVWFSASATAANGWEIYKSRFMTTDGRIQDTGNKNVSHTEGQGFAMLMAVHYDDRIAFDNLWNWTQSHLRNTTSGLFYWRYDPSAANPVVDKNNASDGDVLIAWALLKAGNKWQDNRYLQASDSIQKAIIASNIIQFAGRTVMLPGAYGFNKNSYVILNPSYFLFPAWRDFANRSHLQVWRQLIDDSLSLVGEMRFGQVGLPTDWAALNADGSMAPATAWPSRFSYDAIRIPLYLYWYDAKTTALVPFQLYWRNYPRLTTPAWVDVLSSNTATYNMQGGLLAVRDLTMGNLDGLSDLPGASEDYYSSSLRLLVMLARGK

SEQ ID NO: 8 - protein, 426 aa, synthetic construct
MPLSYLDKNPVIDSKKHALRKKLFLSCAYFGLSLACLSSNAWASVEPLSVNGNKIYAGEKAKSFAGNSLFWSNNGWGGEKFYTADTVASLKKDWKSSIVRAAMGVQESGGYLQDPAGNKAKVERVVDAAIANDMYAIIGWHSHSAENNRSEAIRFFQEMARKYGNKPNVIYEIYNEPLQVSWSNTIKPYAEAVISAIRAIDPDNLIIVGTPSWSQNVDEASRDPINAKNIAYTLHFYAGTHGESLRNKARQALNNGIALFVTEWGTVNADGNGGVNQTETDAWVTFMRDNNISNANWALNDKNEGASTYYPDSKNLTESGKKVKSIIQSWPYKAGSAASATTDPSTDTTTDTTVDEPTTTDTPATADCANANVYPNWVSKDWAGGQPTHNEAGQSIVYKGNLYTANWYTASVPGSDSSWTQVGSCN

SEQ ID NO: 9 - protein, 281 aa, synthetic construct
MNGERIIAFQGRPGAYSDLACRQARPGWTTLPCQTFAQTIAAVHDGRAELAMLACENSLAGRVPDIHALLPEAGLFIVGEHFQRVEHCLLGIPGSTLADARRIHTHPVAMAQVRGIITELGLDPVVEFDTAGAAEMVREWGRKEDVAVASALAAELNGLEILRRNVEDATHNTTRFYIASRRPATLPPPGPGFMTTLLFRVNNQPGALYKALGGLATAGVNMTRLESYMLEGSFSATQFLMDVEGHPEAPPLARALDELSFFSEQQEILGVYPASPFRRKP

SEQ ID NO: 10 - protein, 320 aa, synthetic construct
MPHAETNRRTGQQETTMSLDNASRPSRSGRDELVPSRYAVKVGEIDVLVISDGVLPIPTPVLATNADPAVRTAWLDDMFLPTDVLHWPLNVVLVRSGGQAILVDAGLGVEFPDFPRAGQTVSRLEAAGVDLASVTDVVLTHMHMDHIGGLLADGVKDRLRPDLRIHVAAAEVKFWEAPDFSHASMPSTVPPVLRRTAKRFMAEYQSQLRQFDEDYEVAPGVVVTRTGGHTPGHSVVRLASGGDRLTFAGDAVFQVGFDHPDWHNGFEHDPEEAARVRVRLLRELAATREPLVATHLPFPSVCHVAADGDVFRWVPVVWDS

SEQ ID NO: 11 - protein, 983 aa, synthetic construct
MAVSSNNAEMADDWATFDPAWYRARYAAMLDLMDIPVEQTREFHDAHGAALGHAPNPFFDEEWYRATYPDVAAQVTAGTWRSGFDHYLNAGARTHNPHWLFDERAYLAACPDITPATLDAGGYRNAYDHYLRVGDGEMRGGSCFFDPETCIALLAECDQDGTRRPFAAYLRRGMDALPCRSVSLYFDAEWYVQAYPEAHAEITQGLWRSALHHYLCNPTPQAFDPGPFFSESFYAMVNPDVQAAIEAGALRNGYAHFLSDGVHEQRKPCSTLDLAHYMRDPGVQADIAGRRARDGLGHYLAARPDLKPPPPPQVTEDQARALFRHMCAARLPLLLDGRIDFTTDAPPALSVIIVAHDQFELTMSTLASLRANYHGPMQVVVVDSGSRDGVAGIEDHVRGIEVLRFAGNIGFVRGCNAALRRVRAPATLYLNNDVDLQYGAIANALSRLMADETTGAVGGRIIRTHGMLQEAGSMIWRDGSVQGYMRDAHPCVPEAGFVRAVDFCSGVFLMVRTEVAQVLDGFDESYAPAYFEETDLCVRIRALGYRILYDPAVCLVHYECGTSDGTSASRLIARNSDLFIRRHASGLRRRLLRHAPNEARARHADDGRHILFIEDRLPLRHLGSGFTRSHDIVTTLAGLGYHVTIYPIFRPIEDAATRAAAFPENVEVIHDRELPDLPAFLRARSGCFDAIWIARTHNAARVASILNDAASYIPAGRIVVDTEALAACRDMEYDRLHAITPSRPLDARLADELRPLFVAQRVVAVNTAEADLLRGAGFDNVSVLGHVQAPRATGPGWAARRDILFLGAVHDMASPNLDSLAWFSGQVLPLLVAQLGPDIRFSVCGHASPRVDLGPLRHHPNVRMLGRVADTAPVYDHHRVFVAPTRYGAGIAYKLHEAAANGLPVVGSHLLCRQAGWRDGQDMLCASITDPADFARQVVRLYHDQVLWDTVRDNALTRITTEHAPQDYRNAVATIMDAVFTRG

SEQ ID NO: 12 - protein, 236 aa, synthetic construct
MVGWTIDLLAKLHDLENAATKHDLLSIYLDAVLSVGNVHVTIVELNRIEDPKENFIHVGYPTEWVNFYIENNYIVSDPIIKKSRFMSHPYFWHEIRNINKSEKKIIRDVSEFGIKKGLTIPVHTHDRLIYAICFAFTDKNVDQEIELYLRALSNFFIAGYKKLDEPTDMSPPILTPREKECLRWTAKGKSSWETGMIVGVSERTVNFHINNALLKLKCTNRIMGVVRAICAGLIEL

SEQ ID NO: 13 - protein, 222 aa, synthetic construct
MNDPLIQQEESGSMIEVVTVENAHWCGTALAEQFKFRYRHFVANEQWEVPFYKGMEYDQFDTPAAVYLVWRDVAGVVRGMIRLLPTNRPYMLETLWPDMMPDPIVLPGPTVWEITRFGVERNLSLSLRKQISAELILACIEFSVLNDIHTYLFLTAWGVLKRIVPGAGVDAQIHSRKTLPSGHDVASAVVPVSQAVLDKARAKLNIHYAVLDNNSIERQHAA

SEQ ID NO: 14 - protein, 89 aa, synthetic construct
MRPFANGELALHRRNNHQYGNRKIKHKRTRTLFYREGDIEYLAQQLFSQHYPFRKWDDRPDSISGGPTQEEKDKFKDIARQQLSGWHPV

SEQ ID NO: 15 - protein, 398 aa, synthetic construct
MRLRMVLLATALGAAPFATAMATTITGPYVDIGGGYDLTQTQHAHGFDKNQYENNANTAGYLDATDNARLLKEAHSRERMEHGDGWTGFATFGWGFGNGLRAEIEGDYNWSALTGYNSVSGSAYGNNHQSGKSSGSDRSYGGFVNVLYDIDLKRLFNIDVPVTPFVGVGAGYLWQNVDASTSVTRYLNVRQNGTNGSFAYQGIVGAAYDIPGVPGLQMTTEYRMIGQVESFAMGNISQTGGGDRTLSYDHRFNHQFVGVRYAFNHAPPPPPPAPAVAPPAPSAARTYLVFFDWDGAVLTDRARGIVAEAAQASTHVQTTRIEVNGYTDNTSAHPGPRGEKYNLGLSMRRADSVKAELIRDGVPAGGIDIHGYGEAHPLVVTQPDTREPQNRRVEIILH